(19) United States
(12) Patent Application Publication
(10) Pub. No.: US 2007/0184520 A1
(43) Pub. Date: Aug. 9, 2007

(54) Reduction of spontaneous mutation rates in cells

(76) Inventors: Heike Strobel, Vienna (AT); Wolfgang Schoenfeld, Vienna (AT)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/590,829

(22) PCT Filed: Feb. 26, 2005
(86) PCT No.: PCT/EP05/02067

§ 371 Date: Jan. 29, 2007

(30) Foreign Application Priority Data
Feb. 26, 2004 (EP) 04360019.6

(51) Int. Cl. C12P 21/06; C12N 15/74; C12N 15/09; C12N 1/18; C12N 1/21

(52) U.S. Cl. 435/69.1; 435/455; 435/483; 435/471; 435/325; 435/252.3; 435/254.2; 435/252.33

(57) ABSTRACT

The present invention relates to processes for reducing the spontaneous mutation frequencies in cells or organisms and for producing such cells and organisms, to cells and/or organisms with reduced spontaneous mutation frequencies and to processes for the generation of expression systems for proteins, for the production of proteins and for the production of fermentation products by using cells with reduced spontaneous mutation frequencies.

The present invention relates to processes for reducing the spontaneous mutation frequencies in cells or organisms and for producing such cells and organisms, to cells and/or organisms with reduced spontaneous mutation frequencies and to processes for the generation of expression systems for proteins, for the production of proteins and for the production of fermentation products by using cells with reduced spontaneous mutation frequencies.

Mutation is a characteristic of living systems and provides the material for naturals selection. Organisms such as bacteria constantly undergo spontaneous mutations. The rates of mutations vary greatly according to the organism, the size of, for example, a particular gene and the sensitivity of a gene to inactivation by base pair per substitutions. In Escherichia coli the value of mutations per genome per genome replication is μ_{g}=0.0025 and the mutation rate per base pair per replication is μ_{b}=5.4×10⁻¹⁰ within a genome size of 4.6×10⁶ base pairs. The rate of even a single well-defined pathway such as G-C→A-T can vary by more than 2000-fold at different sites within a single gene, presumably under the still largely mysterious influences of local DNA sequence and structure of the DNA. Both the kind of mutations and the processes that generate them are diverse and only partially discovered.

Mutations are also important in multicellular organisms. However, here two different kinds of changes in the genetic material have to be distinguished, namely mutations in gametes, i.e. germ line mutations, and changes in body cells, i.e. somatic mutations. Whereas germline mutations are passed on to the offspring of the organism, somatic mutations are not. However, somatic mutations can be important as they contribute to the development of cancer. In detection of germline mutations, in particular in humans and measurement of human mutation rates, there is the problem of diploidy. Most forward mutations are recessive and so will not be detected unless a zygote gets two copies of the mutant allele. Reversions are generally much less frequent because there are a lot more ways to mutate a gene than there are to reverse an existing mutation. From in vivo studies using human cells in vitro it is estimated that the overall human mutation rate is very similar to those measured in various prokaryotic and eukaryotic microorganisms.

Mutations, such as heritable alterations in the genetic material may be gross alterations, in particular at the level of the chromosome, or point alterations which may not be visible as cytological abnormalities. Point mutations include base pair substitutions such as transitions and transversions and frameshift mutations. The consequences of base substitution mutations in protein coding regions of a gene depend on the type of substitution and its location. Such base pair substitutions might be silent, i.e. do not result in a new amino acid residue in the protein sequence. However, they can also result in an amino acid substitution. Such missense-mutations may have very serious consequences, as in the case of sickle-cell anemia, mild consequences or no consequences at all. Finally, base substitutions in a protein coding region may mutate an amino acid codon to a termination codon or vice versa. The formerly type, which results in a prematurely shortened protein, is referred as a nonsense mutation. Base substitution mutations may also occur in sequences involved in the regulation of the expression of a gene such as in promoters or 5′-regulatory regions of genes or in introns and may effect their transcription, translation or splicing. Many of the β-thalassemias are the result of this types of non-structural mutations that effect the level of expression of the globin genes. Frameshift mutations result from the insertion or deletion of one or more (but not in multiples of three) nucleotides in the coding region of a gene. This causes an alteration of the reading frame. A mutation of this sort changes all the amino acids downstream and is very likely to create an non-functional product since it may differ greatly from the normal protein.

Spontaneous mutations can occur as a result of natural processes in the cell which can be distinguished from induced mutations, i.e. mutations that occur as a result of the interaction of DNA with an outside agent or mutagen. An important source of spontaneous mutations are mistakes in DNA replication, for example due to the incorrect action of a DNA polymerase. The frequency at which DNA polymerases make mistakes will influence this spontaneous mutation frequency whereby it has been observed that different DNA polymerases vary in there accuracy. One major factor affecting the DNA polymerase accuracy is the presence of a proofreading 3′-5′ exonuclease which will remove incorrectly paired bases inserted by the polymerase. The function of the 3′-5′ exonuclease is to prevent misincorporation during DNA replication and to prevent mutations.

Another major source of spontaneous mutations are structural alterations of the bases of nucleic acids called tautomerization. Bases are capable of existing in two forms between which they interconvert. For example, guanine can exist in keto and enol forms. The various tautomer forms of the bases have different pairing properties. If during DNA replication G is in the enol form, the DNA polymerase will add T across from it instead of the normal C. Therefore, tautomerization is responsible for transition mutations. Another mutagenic process occurring in cells is spontaneous base degradation. The deamination of cytosine to uracil happens at a significant rate in cells. Deamination can be repaired by a specific repair process which detects uracil, not normally present in DNA; otherwise the U will cause A to be inserted opposite it and can cause a C:G to T:A transition when the DNA is replicated. A third type of spontaneous DNA damage that occurs frequently is damage to the bases by free radicals of oxygen. These arise in cells as a result of oxidative metabolism and also are formed by physical agents such as radiation. An important oxidation product is 8-hydroxyguanine, which mispairs with adenine, resulting in G:C to T:A transversions. Still another type of spontaneous DNA damage is alkylation, the addition of alkyl groups to the bases or backbone of DNA. Alkylation can occur through reaction of compounds such as S-adenosyl methionine with DNA. Alkylated bases may be subject to a spontaneous break down or mispairing.

Furthermore, spontaneous frameshift mutations can also arise by a mechanism called “slipped mispairing” between the template strand and the newly synthesized strand during DNA replication.

Spontaneous mutations arise randomly at any site of the genome, whereby most of the spontaneously occurring mutations are detrimental to the organism affected and the probability of the arise of an advantageous mutation is very low. Therefore, organisms have evolved mechanisms to protect themselves from excessive mutation rates. These protective mechanisms recognize and correct mismatches that have occurred in DNA as a result of replication or spontaneous deamination of the DNA and recognize and remove potentially mutagenic changes that have occurred as a result of the reaction of DNA with exogenous or endogenous mutagens.

In particular in biotechnology processes such as fermentation processes mutations are highly undesired. Since mutations can occur in any part of the genome of the fermenting cell they can affect the formation or the composition of the fermentation product. If, for example, the fermentation product is a protein, a mutation of the nucleic acid sequence encoding the protein can lead to a protein variant with an altered amino acid sequence. Even if only a minor portion of the fermenting cells is affected by the mutation this can result in a final protein preparation which is contaminated with that variant. This can have dramatic unforeseeable consequences in such cases where the protein shall be used for the therapy of a disease in humans, for example, if the antigenic properties of the protein are altered. The arise of mutations within the population of fermenting cells can also influence the rate of the product formation, if the mutation for example affects an upstream regulatory pathway such as an enzyme involved in the formation of the fermentation product or a regulatory unit which controls the expression of the fermentation product desired.

Furthermore, even if mutations are rare events affecting only a minor portion of a population of fermenting cells they can spread quickly in such a population if they confer the cells affected a selective advantage in comparison to non-mutated cells. In particular in continuous cultures with a constant removal of cells to maintain a steady-state this can lead with advancing duration of the cultivation to a progressive decrease of the non-mutated cells relative to the mutated cells.

Another reason, why mutations in particular during fermentation are highly undesired is the phenomenon of the so-called stationary-phase mutation, which is also called adaptive mutation. When populations of microorganisms are exposed to nonlethal selections such as that occurring during the stationary-phase of the fermentation, mutations that relieve the selective pressure arise with high frequency (Cairns et al., Genetics, 128 (1991), 695-701). Although it originally seemed that only useful mutations appeared, it is now clear that selected mutations are accompanied by non-selected mutations, i.e., the process is not directed to useful genes (Foster, J. Bacteriol., 179 (1997), 1550-1554). Most research on adaptive mutation has focused on a strain of Escherichia coli that cannot utilize lactose (Lac⁻) but that readily reverts to lactose utilization (Lac⁺) when lactose is its only carbon source. The process that produces adaptive mutation is not the same as that, which produces Lae⁺ mutations during normal growth. Unlike growth-dependent mutations, almost all adaptive Lac⁺ mutations are dependent on recombination functions, such as the homologous recombination function of the RecBCD double-strand break (DSB) repair system of E. coli (Cairns et al., Genetics, 128 (1991), 695-701; Foster, Annu. Rev. Microbiol. 47 (1993), 467-504; Harris et al., Science, 264 (1994), 258-260).

Therefore, the technical problem underlying the present invention is to provide means and methods for the production of a fermentation product, such as a protein, by a cell or an organism, wherein the producing cell or organism is protected and/or stabilised against spontaneously occurring mutations, in particular during the stationary-phase of the fermentation process, and whereby both the rate of the formation and the composition of the fermentation product, in particular the protein, is secured and protected on a long-term scale.

The present invention solves this technical problem by providing a process for reducing the spontaneous mutation frequencies in a cell or an organism by introducing at least two mutations, whose combined actions lead to at least two enhanced cellular DNA repair mechanisms, into the cell or organism.

The present invention solves the underlying technical problem also by providing a process for producing a cell or an organism with reduced spontaneous mutation frequencies by introducing at least two mutations, whose combined action lead to at least two enhanced cellular DNA repair mechanisms, into at least one cell of the organism and regenerating the organism therefrom, if the organism is a multi-cellular organism.

According to the invention the capability of cellular DNA repair mechanisms to correct spontaneously occurring mutations is greatly enhanced, whereby at least two different mutations, which affect different repair systems are introduced into the cell. Advantageously, the enhanced capability of the cellular DNA repair mechanisms to correct spontaneously occurring mutations obtained by the inventive introduction of at least two different mutations leads to an reduced frequency of stably inherited mutation in the cell and thus to an over-all reduced mutation rate.

Thus, according to the invention it was surprisingly found that by altering certain cellular DNA repair mechanisms, in particular by the introduction of such specific mutations, that enhance the capability of these DNA repair systems to repair spontaneously occurring mutations more efficiently, the spontaneous mutation rates of wild-type cells can dramatically be decreased. For example, according to the invention it was surprisingly found, that overexpression of the MutS protein leads during the growth phase to a significant reduced mutability of the host cell. This is, however, in contrast to results described in the state of art. According to U.S. Pat. No. 6,656,736 overexpression of wild-type or mutant MMR proteins from yeast or other organisms result in a defective mismatch repair system, whereby yeast cells with such a defective mismatch repair system are hypermutable. Furthermore, a bacterial strain carrying the deletion dinB10 and the antimutator allele dnaE911 shows a 10-fold reduced mutability in comparison to the corresponding wild-type strain. An additional overexpression of the protein MutL involved in the mismatch repair system drops the mutability up to a factor of 50. In another system it could be shown that the reversion of specific frameshift mutations could be even decreased by a factor of 1,000 In this way not only growth-dependent mutation rates can be significantly decreased, but also the mutation rates during adaptive mutation, i.e. the stationary-phase dependent mutation rates. Surprisingly the effects of several mutations on the spontaneous mutation rate of the host cell observed appear to combine in a synergistic, but not in an additive manner.

Furthermore, according to the invention it was surprisingly found that the introduction of these mutations not only leads to an enhanced capability of the cellular DNA repair mechanisms to correct spontaneously occurring mutations, but advantageously also to a greatly increased cellular viability. For example, it was found that overexpression of the MutL protein in a fermenting bacterial strain increased the cellular viability by a factor of about 10³.

The inventive reduction of the spontaneous mutation rates in cells and the inventive increase in cellular viability by introducing several mutations enhancing the capability of cellular DNA repair mechanisms to correct spontaneously occurring mutations is in particular of great value for such cells or strains which are used for the expression and/or generation of fermentation products such as proteins. By the use of such cells for example the amino acid sequence of protein products obtained can advantageously maintained unchanged over many generations of the producing cell or organism. Protein preparations obtained by the use of such cells are not contaminated by protein variants due to mutation and therefore do not cause any problems upon application as therapeutic agents and do not lead to undesired effects in the recipient body. The use of such inventive cells with enhanced capabilities of cellular DNA repair mechanisms is in particular useful for the production of recombinant proteins.

However, the use of the inventive cells with an enhanced capability of cellular DNA repair mechanisms is not restricted to the production of proteins by fermenting cells. In principal, the inventive cells can be used for all kinds of fermentation products such as antibiotics, organic acids etc. The greatly reduced spontaneous mutation rates in these cells provide not only for the maintenance of the integrity of the product composition on a long-term scale, but also for the maintenance of high rates of product formation since due to the decreased mutation rates in the host cells also those factors which control the effectiveness of the rate of product formation will remain stable and unchanged.

In the context of the present invention the term “mutation that leads to an enhanced capability of a cellular DNA repair mechanism” means any heritable alteration of that part of the genome of a cell which encodes proteins or enzymes involved in at least one specific DNA repair mechanism or any heritable alteration of that part of the genome which is involved in the regulation of the expression of such constituents of a cellular DNA repair mechanism, that leads to an enhanced recognition and correction of genomwide errors within a cell or organism that have occurred in DNA as a result of replication or spontaneous deamination of the DNA or any other natural processes leading to spontaneously occurring mutations. A “mutation that leads to an enhanced capability of a cellular DNA repair mechanism” therefore provides for a more efficient and more accurate correction of spontaneously occurring errors within the genome of a given cell or organism.

A “mutation that leads to an enhanced capability of a cellular DNA repair mechanism” therefore leads to a reduced frequency of stably inherited mutations within a population, i.e. to a reduced mutation frequency of that population. In the context of the present invention “mutation frequency” is defined as the ratio of the number of mutants to the total number of individuals of a population. A “mutation that leads to an enhanced capability of a cellular DNA repair mechanism” also leads to a reduced mutation rate, which is defined as the probability, with which a given gene will mutate during replication and with which this mutation of the gene will be stably inherited. Furthermore, in the context of the present invention a mutation that leads to an enhanced capability of a cellular DNA repair mechanism also leads to a reduction of transposon-mediated mutagenesis.

A cell or organism therefore exhibits due to the presence of a mutation that leads to an enhanced capability of a cellular repair mechanism a very low spontaneous mutation rate, which is in particular considerably lower than that of a corresponding cell or organism without that mutation. Mutations that lead to an enhanced capability of a cellular DNA repair mechanism include, without being restricted to, deletions of structural genes encoding enzymes involved in cellular DNA repair, for example a deletion of the structural gene of an error-prone DNA polymerase; substitutions, deletions, inversions and/or addition of bases in a structural gene encoding an enzyme involved in cellular DNA repair, which, for example can lead to an antimutator phenotype or an improved fidelity of a DNA polymerase, overexpression of a protein, which becomes limiting during a certain phase of growth, differentiation or propagation of a cell or organism, reduced expression of a protein, for example an error-prone DNA polymerase, etc. in the context of the present invention, each individual mutation that leads to an enhanced capability of a cellular DNA repair mechanism, can, however, also lead to an enhanced capability of a second or third cellular DNA repair mechanism. Furthermore, each individual mutation that leads to an enhanced capability of a cellular DNA repair mechanism, can also lead to an enhanced cellular viability.

In the context of the present invention the term “two mutations, whose combined actions lead to at least two enhanced cellular DNA repair mechanism” means that both mutations affect at least two different DNA repair mechanisms such that the capability of these DNA repair systems to repair spontaneously occurring genomwide mutations more efficiently and/or more accurately is enhanced in comparison to the non-mutated DNA repair systems.

In the context of the present invention, the term “cellular DNA repair mechanism” means an enzymatic mechanism or system by which a cell or organism is able to recognize and correct any error in the genome that occurs by DNA replication and/or that is due to base alterations and base damage. In preferred embodiments of the invention these cellular DNA repair mechanisms include the mismatch repair system, the post-replicative (recombinational) repair system and the SOS repair system. Mutations that lead to an enhanced mismatch repair are in particular those, which overcome a situation where one of the proteins involved in mismatch repair becomes limited.

The “mismatch repair system” (MMR) accounts for 99% of all repair events in cells. This system is the largest contributor to replication fidelity in E. coli whereby it also acts in other DNA repair pathways by the involvement of its component proteins in transcription-coupled DNA repair and very-short-patch repair. MMR also enforces genetic stability by suppressing recombination between imprecise homologies that can otherwise cause genome rearrangements. Mismatch repair also promotes genetic stability by editing transposon excision. Homologs of the E. coli MMR proteins perform similar functions in other bacteria, yeast, mice and humans (Modrich, Science, 266 (1994), 1959-1960; Radman et al., Phil. Trans. R. Soc. London, 347 (1995), 97-103). In E. coli the gene products of mutH, mutL, mutS and mutU are involved in MMR. The system recognises the newly synthesized strand rather than the parent strand because of methylation. The mutS gene product recognizes and binds to DNA base mismatches. The mutL gene product interacts with MutS after mismatch binding and is thought to coordinate MutS with MutH. The MutH exonuclease then nicks the unmetylated new DNA strand. The MutU helicase enters DNA at the single-strand nick and displaces the nicked strand. Mutations that lead to an enhanced mismatch repair are in particular those, which overcome a situation where one of the proteins involved in mismatch repair becomes limited.

Post-replicative repair is a system, wherein lesions in the DNA are repaired in the following replication round. Due to replication of the damaged DNA in the daughter strands gaps are created. The missing genetic information is filled by corresponding DNA regions from the parental strand by recombination. Replication of damage-containing DNA, a process, which is also called translesion synthesis, is a major source of point mutations. Recently, this process has gained much understanding as the DNA polymerases involved have been identified (Friedberg et al., Proc. Natl. Acad. Sci. USA, 97 (2000), 5681-5683).

The SOS response is a set of cellular responses induced by the exposure of cells to a variety of genotoxic and metabolic stresses which generally interfere with DNA replication. Regulation of the SOS system is mediated by the LexA and RecA proteins LexA acts as a repressor of about 30 different genes including recA and lexA. The SOS inducing signal is single-stranded DNA, to which RecA binds and becomes activated as a coprotease (RecA*). RecA* promotes proteolytic self-cleavage of the LexA repressor and of some phage repressors, thus derepressing the SOS regulon. Escherichia coli has at least three SOS inducible polymerases: polymerase II (Pol II), polymerase IV (Pol IV) and polymerase V (PoIV).

According to the invention at least two mutations are selected from a mutation leading to an up-regulation of the expression of the MutL protein or a homologous protein thereof, from a mutation leading to an up-regulation of the expression of the MutS protein or a homologous protein thereof, an antimutator allele of a gene encoding DNA polymerase IV or a homologous protein thereof and an antimutator allele of a gene encoding a subunit of DNA polymerase III or a homologous protein thereof. In the context of the present invention “antimutator allele” means an allele which causes a decrease in the spontaneous mutation frequencies in a cell or organism in comparison to the corresponding wild-type cell.

In a particular preferred embodiment of the invention, the upregulation of the expression of MutL or a homologous protein thereof and the expression of MutS or a homologous protein thereof, respectively, is achieved by introducing a vector within the cell, wherein the vector comprises the mutL gene or a gene encoding a homologous protein of MutL and the mutS gene or a gene encoding a homologous protein of MutS, respectively, under the functional control of one or more regulatory units allowing an overexpression of the respective Mut protein. Preferably, the vector is a multicopy plasmid.

Preferably, the regulatory unit is an inducible or constitutive promoter.

In another preferred embodiment of the invention the up-regulation of the expression of MutL or a homologous protein thereof and the expression of MutS or a homologous protein thereof, respectively, is achieved by alterations of those regulatory units which direct the expression of the native Mut protein of the host cell or host organism such that higher amounts of the native Mut protein are produced in the cell than that usually observed in the cell. This means, that regulatory units directing the transcription of the chromosomally located native nucleotide sequence encoding the respective Mut protein into a complementary RNA sequence and/or and the translation of the coding RNA sequence thus obtained into the polypeptide chain of the Mut protein are mutated or replaced by other suitable homologous or heterologous regulatory units such that a higher production of the native Mut protein than that observed in a corresponding wild-type cell or organism is obtained.

In another embodiment of the invention an overexpression of the respective Mut protein is achieved by the introduction of one or more additional copies of the respective mut gene under the functional control of appropriate regulatory units into the chromosome(s) of the host cell, whereby the additional mut gene(s) can either be the native gene(s) or (an) heterologous gene(s) derived from another species.

In a preferred embodiment of the invention, the antimutator allele of the gene encoding DNA polymerase IV or a homologous protein thereof is dinB10 which is in fact a deletion of the dinB-gene. The dinB-encoded DNA polymerase IV (Pol IV) belongs to the recently identified Y-family or UmuC/DinB nucleotidyltransferase family of error-prone DNA polymerases. This family is represented by the UmuC, DinB, Rad30 and Rev1 subfamilies (Gerlach et al., Proc. Natl. Acad. Sci. USA, 96 81999), 11922-11927). The dinB-encoded DNA polymerase IV of E. coli has been shown to be involved in spontaneous mutagenesis and in the replication of a damage-containing DNA, a process termed translesion synthesis (TLS), a major source of point mutations. Like DNA polymerase V, Pol IV is also induced as part of the SOS response to DNA damage.

In another preferred embodiment of the invention, an antimutator allele of a gene encoding a subunit of DNA polymerase III is used, which is preferably dnaE911. DNA polymerase III holoenzyme (Pol III HE) accounts for more than 90% of cellular DNA synthesis and is also required for the major post-replicative mismatch correction pathway. Polymerase III holoenzyme was shown to effectively carry out translesion DNA synthesis past abasic sites. The dnaE gene of E. coli encodes the a subunit of DNA polymerase III holoenzyme, which provides the polymerase activity. The a subunit therefore is one of the main determinants of fidelity. From Fijalkowska and Schaaper, J. Bact., 177 (1995), 5979-5986, several dnaE antimutator alleles are known, which suppress the elevated mutability of a mismatch repair defective mutL strain.

According to the invention, the combined action of the mutations dinB10 and dnaE911 reduces the spontaneous mutation frequency of a cell in comparison to a wild-type cell or wild-type organism, at least 10-fold. In another preferred embodiment of the invention, the combined action of dinB10, dnaE911 and the overexpressed MutL protein reduces the spontaneous mutation frequency in a cell in comparison to a wild-type cell or wild-type organism at least 50-fold.

The at least two mutations, whose combined action lead to enhanced cellular DNA repair mechanisms and/or an enhanced cellular viability can be introduced by any known procedure into a cell, in particular by either mutagenesis of a given cell in order to mutate a gene known to be involved in one of the cellular DNA repair systems or by introduction of already known mutations or alleles into that cells.

A number of different mutagenesis methods exist, including, but not restricted to random mutagenesis, site-directed mutagenesis, oligonucleotide cassette mutagenesis, or point mutagenesis by error-prone PCR. Random mutagenesis, for example, entails the generation of a large number of randomly distributed, nucleotide substitution mutations in cloned DNA fragments by treatment with chemicals such as nitrous acid, hydrazine, etc. Error-prone PCR has been developed to introduce random point mutations into cloned genes. Modifications that decrease the fidelity of the PCR reaction include increasing the concentration of MgCl₂, adding MnCl₂, or altering the relative concentrations of the four dNTPs. These traditional mutagenesis methods focus on the alteration of individual genes having discrete and selectable phenotypes. The general strategy is to clone a gene, establish an assay by which the gene and/or its function can be monitored, mutate selected positions in the gene and select variants of the gene for altering the function of the gene. A variant having altered functions can then be introduced and expressed in a desired cell type. Repetitive cycles of mutagenesis methods can be carried out to obtain desirable functions of the gene. Another approach to alter the function of a gene is by recombination by using one of the numerous well-established systems.

Of course already existing alleles or mutations can be used for the inventive purposes. Such existing alleles or mutations can be introduced into the cell by any of the known procedures. According to the invention the introduction of the at least two mutations into a cell can be effected by any known appropriate method including, but not restricted to, transformation, conjugation, transduction, sexduction, infection and/or electroporation.

In the context of the present invention the term “transformation” means the uptake of an isolated, preferably purified, nucleic acid molecule from the environment by a cell, for example a microbial cell “Conjugation” means the plasmid-mediated transfer of a bacterial plasmid from one bacterial cell into another bacterial cell through cell-to-cell-contact. The transfer machinery involved is usually encoded by plasmids or conjugative transposons. Examples of such plasmids are conjugative plasmids or helper plasmids. Conjugation is one of the major routes for genetic exchange between different phylogenetic groups of prokaryote cells and between prokaryotes and eukaryotes. “Transduction” means the transfer of a nucleic acid molecule from one bacterial cell into another bacterial cell by a bacteriophage, which comprises the release of a bacteriophage from one cell and the subsequent infection of the other cell. There are two types of transduction. A spezialized transduction may occur during the lysogenic life cycle of a temperate bacteriophage, whereby genetic material of bacteria can replace a part of the bacteriophage genome. This piece of bacterial DNA replicates as a part of the phage genome can be transferred by the phage into another recipient cell. In case of a generalized transduction the entire genome of a lytic phage can be replaced by bacterial DNA. “Electroporation” is a process where cells are mixed with nucleic acid molecules and then briefly exposed to pulses of high electrical voltage. The cell membrane of the host cell is penetrable thereby allowing foreign nucleic acids to enter the host cell.

The cell used in the inventive process for reducing the spontaneous mutation rates and in the inventive process for generating a cell or organism with reduced spontaneous mutation rate can be any prokaryotic or eukaryotic cell.

The terms “eukaryotic cell” and “eukaryotic host cell” include any cells that have a membrane-bound nucleus and organelles and genetic material organized in chromosomes in which the DNA is combined with histones. The cytoskeleton is another feature unique to the eukaryotes, which is a network of protein filaments, mostly actin and tubulin, which are anchored to the cell membrane and criss-cross the periphery of the cell. “Eukaryotes” comprise the taxonomic kingdoms Protista, Fungi, Plantae and Animalia. Eukaryotes can be unicellular organisms such as protists, for example paramecium and amoebae, or multicellular organisms such as diverse fungi, animals and plants. In a preferred embodiment of the inventive processes for reducing spontaneous mutation rates animal cells, plant cells or fungal cells are used.

The terms “prokaryotic cell” and “prokaryotic host cell” include any cell, in which the genome is freely present within the cytoplasm as a circular structure, i.e. a cell, in which the genome is not surrounded by a nuclear membrane. A prokaryotic cell is further characterized in that it is not necessarily dependant on oxygen and its ribosomes are smaller than that of eukaryotic cells Prokaryotic cells include archaebacteria and eubacteria. In dependence on the composition of the cell wail eubacteria can be divided into gram-positive bacteria, gram-negative bacteria and cyanobacteria. In a preferred embodiment of the inventive process for reducing spontaneous mutation rates or for generating corresponding organisms the prokaryotic cell used is a cell of an archaebacterium or an eubacterium, whereby particularly preferred the prokaryotic cell is a gram-negative bacterium, a gram-positive bacterium or an cyanobacterium.

The present invention also relates to a cell with a reduced spontaneous mutation frequency and/or an enhanced cellular viability, which is obtainable by the inventive process for reducing the spontaneous mutation frequencies in a cell or an organism or by the inventive process for producing a cell or an organism with reduced spontaneous mutation frequencies, wherein the cell comprises at least two mutations, whose combined actions lead to at least two enhanced cellular DNA repair mechanisms. In a preferred embodiment of the invention the cell is a bacterial cell, for example a gram-negative bacterium such as E. coli or a gram-positive bacterium such as Bacillus subtilis, a fungal cell, plant cell or animal cell such an insect cell or mammalian cell.

In a particular preferred embodiment of the invention the bacterium is E. coli MG1655dinB10 containing plasmid pmutL, E. coli MG1655dinB10 mutL::tet containing plasmid pmutL, E. coli MG1655 dnaE zae::cm containing plasmid pmutL, E. coli MG1655 dnaE zae::cm mutL::tet containing plasmid pmutL, E. coli MG1655dinB10 dnaE zae::cm, E. coli MG1655dinB10 dnaE zae::cm mutL::tet, E. coli MG1655dinB10 dnaE zae::cm containing plasmid pmutL or E. coli MG1655dinB10 dnaE zae::cm mutL::tet containing plasmid pmutL.

The present invention also relates to the use of the inventive cells with reduced spontaneous mutation rates and/or enhanced cellular viability for the generation of organisms with reduced spontaneous mutation rates, the use of the inventive cells as host cells for the expression of a protein, the use of the inventive cells as fermenting organisms for the production of fermentation products, the use of the inventive cells as model system for studying the effects of candidate drugs, the use of the inventive cells as model system for studying human, animal or plant diseases, and the use of the inventive cells for the generation, in particular breeding or cultivation of transgenic organisms.

The person skilled in the art knows numerous procedures how a complete multi-cellular organism, in particular a plant or an animal, can be generated from single cells. Multi-cellular organisms which are generated from the inventive cells are likewise characterized by overall reduced spontaneous mutation rates.

The present invention also relates to an organism with reduced spontaneous mutation frequencies, which is obtainable by the inventive process for reducing the spontaneous mutation frequencies in a cell or an organism or by the inventive process for producing a cell or an organism with reduced spontaneous mutation frequencies, wherein the cells of the organism comprise at least two mutations, whose combined actions lead to at least two enhanced cellular DNA repair mechanisms and/or enhanced cellular viability. In a preferred embodiment of the invention the multi-cellular organism is a fungus such as Saccharomyces cerevisiae or Aspergillus nidulans, a plant, in particular a plant with agricultural utility such as Zea mays or an animal, in particular a mammal, for example a mammal which can be used as model system for studying a given disease or the effects of drug candidates for the therapeutic treatment of a given disease.

The present invention also relates to the use of the inventive organisms as host for the expression and/or production of proteins such as those which are of economic, medical or agricultural importance, the use of the inventive organisms for breeding or cultivating transgenic organisms, the use of the inventive organisms as model systems for studying diseases and the use of the inventive organisms as model system for studying the effects of candidate drugs.

The technical problem underlying the present invention is also solved by providing a process for the generation of an expression system for a protein wherein the amino acid sequence of the protein is stabilized against spontaneously occurring mutations comprising:
a) inserting a nucleic acid sequence encoding the protein into the genome of a host cell, that contains at least two mutations whose combined actions lead to an enhanced capability of at least two cellular DNA repair mechanisms to repair spontaneously occurring mutations, under the functional control of one or more regulatory units allowing an inducible or constitutive expression of the protein, or
b) inserting a nucleic acid sequence encoding the protein into a vector under the functional control of one or more regulatory units allowing an inducible or constitutive expression of the protein and transferring the vector into a host cell, that contains at least two mutations whose combined actions lead to an enhanced capability of at least two cellular DNA repair mechanisms to repair spontaneously occurring mutations and
c) culturing and/or maintaining the host cell in an appropriate medium.

The present process for the generation of an expression system for a protein is in particular directed to the generation of a host cell in which the expression of the protein can take place and which is characterized by very low spontaneous mutation rates, in particular lower than in other host cells known. The expression system generated by the inventive process therefore secures that the probability is greatly reduced that a nucleotide sequence encoding a given protein and thus the amino acid sequence of that protein will be changed by mutations. The expression system which is provided by the inventive process and based on the inventive host cell with low spontaneous mutation rates and enhanced cellular viability therefore can be used for the expression and generation of such proteins whose integrity shall be secured on a long-term scale. The inventive expression system can especially be used for such proteins which shall be used for therapeutic purposes and in which any changes to the amino acid sequence, for example such that lead to an changed biological activity of the protein or alter the antigenic properties of the protein, could have adverse effects on the therapeutic benefit of the protein. The present process for the generation of an expression system is therefore useful in avoiding even minimal contaminations in a protein preparation to be generated by mutated, aberrant proteins. Advantageously, the expression system which is provided by the inventive process and based on the inventive host cell with low spontaneous mutation rates can be used for the long-term maintenance of nucleotide sequences encoding a particular protein obviating the need of regular examinations by sequencing in order to determine whether the sequence of the nucleotide sequence has altered or not.

In the context of the present invention “expression system” means in particular a cellular system which allows the efficient expression of a given protein, i.e. the production of high amounts of the protein in a cell in particular “expression system” relates to a cellular environment enabling the transcription of a nucleotide sequence encoding the protein desired into a complementary RNA sequence, the splicing of the RNA sequence in order to remove non-coding sequence parts and the translation of the coding RNA sequence parts thus obtained into the polypeptide chain of the protein. “Expression system” can also relate to a cellular environment enabling post-translational processing steps such as glycosylation of the protein or removal of an existing leader sequence from the protein. “Generation of an expression system” therefore means, that the nucleotide sequence encoding the protein desired has to be functionally linked to appropriate regulatory units which control and direct the expression of the encoded gene product in a given cellular environment and that an appropriate host cell has to be provided in which the regulatory units used will function such that an optimum expression of the protein is achieved.

According to the invention the reduced spontaneous mutation rates and enhanced viability in a host cell used in a system for the expression of a protein are obtained by the use of at least two mutations, the combined actions of which lead to an enhanced capability of at least two cellular DNA repair mechanisms to repair spontaneously occurring mutations in any nucleic acid present within the host cell. In particular these at least two mutations enhance the capability of the mismatch repair system, the proof-reading function and/or the SOS repair system to repair spontaneously occurring mutations. In preferred embodiments of the invention these mutations are selected from a mutation leading to an upregulation of the expression of the MutL protein or a homologous protein thereof, a mutation leading to an upregulation of the expression of the MutS protein or a homologous protein thereof, an antimutator allele of a gene encoding DNA polymerase IV or a homologous protein thereof and an antimutator allele of a gene encoding a subunit of DNA polymerase III or a homologous protein thereof.

In one embodiment of the invention the upregulation of the expression of MutL or a homologous protein thereof and the upregulation of the expression of MutS or a homologous protein thereof, respectively, can be achieved by introducing a vector into the host cell, whereby the vector comprises the mutL gene or a gene encoding a homologous protein thereof and the mutS gene or a gene encoding a homologous protein thereof, respectively, under the functional control of one or more regulatory units allowing an overexpression of the respective Mut protein in comparison to a corresponding wild-type host cell. Preferably, the vector used for overexpression of either the MutL or MutS protein is a multi-copy plasmid, which comprises the respective mut gene under the functional control of one or more regulatory units. In a preferred embodiment of the invention the regulatory unit controlling the overexpression of the respective mut gene can be an inducible or constitutive promoter.

In another embodiment of the invention the upregulation of the expression of MutL or a homologous protein thereof and the upregulation of the expression of MutS or a homologous protein thereof, respectively, can be achieved by either introduction of one or more additional copies of the respective mut gene into the chromosome(s) of the cell or by one or more mutations to the regulatory units directing the transcription of the native chromosomally located mut gene such that in the cell in comparison to a corresponding wild-type cell a higher production of the respective Mut protein is effected.

In a preferred embodiment of the invention the antimutator allele of the gene encoding DNA polymerase IV is dinB10. In another preferred embodiment the antimutator allele of the gene encoding the subunit of DNA polymerase III is dnaE911.

According to the invention the expression system generated can be used for the expression of any protein. In the context of the present invention the term “protein” is a molecule that comprises at least two amino acids connected by an amide linkage. Therefore, according to the invention the term “protein” also includes a peptide, for example an oligopeptide, a polypeptide or a part of a naturally occurring protein such as a domain. The amino acid sequence of the protein to be expressed can be that of a naturally occurring protein, i.e. can have a wild-type sequence. Of course the protein to be expressed by the expression system can have an amino acid sequence which is altered in comparison to that of a wild-type protein, for example by a different amino acid composition and/or by a different length. In comparison to the wild-type protein the protein to be expressed can therefore have different amino acid residues at one or more positions. In comparison to the wild-type protein the protein to be expressed can be truncated or elongated. Furthermore, the protein to be expressed can possess characteristics which differ from that of the corresponding wild-type protein. These different features can relate to an altered thermostability, a different substrate specificity, a different activity, altered or new catalytic sites etc., without being restricted thereto. The protein to be expressed can also be a fusion protein comprising two or more individual polypeptides or comprising in addition one or more domains of a second polypeptide. Such alterations or mutations of the protein can be obtained by suitable manipulations of the nucleotide sequence encoding the protein known in the art before the protein-encoding nucleotide sequence is inserted into the genome of the host cell with reduced spontaneous mutation rates or into a vector, which is afterwards introduced into that host cell. In a preferred embodiment of the invention the protein to be expressed is a recombinant protein, i.e. a protein altered by genetic engineering procedures and/or DNA recombination techniques.

In preferred embodiments of the invention the protein to be expressed can be an enzyme, which can be utilized for the industrial production of natural and non-natural compounds. Enzymes or those compounds produced by the help of enzymes can be used for the production of drugs, cosmetics, foodstuffs, etc. The protein to be expressed can also be a substance, that has therapeutic applications in the fields of human and animal health. Important classes of medically important proteins for example include cytokines and growth factors.

According to the inventive process the nucleic acid sequence encoding the protein is inserted either into the genome or into a vector under the functional control of one or more regulatory units. Regulatory units that control and direct the expression of a gene and a gene product, respectively, include, without being restricted to, promoters, ribosome binding sites, enhancers, silencers, polyadenylation sites and/or a 3′-transcription terminators. Regulatory units can also comprise leader or signal sequences directing the protein expressed to a given compartment of the host cell or out of the cell. The use of such regulatory units depends on the type of the host cell used. Regulatory units which can be used in a given prokaryotic or eukaryotic host cell are well known (see for example Sambrook et al., Molecular cloning: A Laboratory Handbook, second edition (1989), Cold Spring Harbor Laboratory Press, NY, USA).

In a preferred embodiment the nucleotide sequence encoding a protein is cloned into a vector. Preferably plasmids, bacteriophages, viruses or cosmids are used as vector. Those skilled in the art also know a large number of suitable vectors for either eukaryotic or prokaryotic host cells which can be used for the introduction of the protein-encoding nucleotide sequence into a given host cell.

Furthermore, the person skilled in the art knows numerous procedures for cloning a protein-encoding nucleotide sequence such, that it is functionally linked to regulatory units, and for introducing nucleotide sequences into a host cell (see for example Sambrook et al., 1989).

A further aspect of the present invention relates to a process for the production of a protein wherein the amino acid sequence of the protein is stabilized against spontaneously occurring mutations comprising:
a) inserting a nucleic acid sequence encoding the protein into the genome of a host cell, that contains at least two mutations whose combined actions lead to an enhanced capability of at least two cellular DNA repair mechanisms to repair spontaneously occurring mutations, under the functional control of one or more regulatory units allowing an inducible or constitutive expression of the protein, or
b) inserting a nucleic acid sequence encoding the protein into a vector under the functional control of one or more regulatory units allowing an inducible or constitutive expression of the protein and transferring the vector into a host cell, that contains at least two mutations whose combined actions lead to an enhanced capability of at least two cellular DNA repair mechanisms to repair spontaneously occurring mutations,
c) culturing the host cell in an appropriate medium under conditions allowing the expression of the protein, and
d) isolating the protein expressed.

Depending on whether the nucleic acid sequence encoding the protein is functionally linked to a leader or signal sequence the protein expressed is transported to a certain cell organelle, a certain cell compartment, the extracellular space or into the medium in which the cells are cultivated. Therefore, the protein can either be accumulated within the cell or is secreted out of the cell. In a preferred embodiment of the inventive process for the protein production the protein is therefore isolated from the medium. In another preferred embodiment of the inventive process for the production of a protein the protein is extracted from the host cell, for example from a certain cell organelle. Also, the person skilled in the art knows numerous protocols for the isolation of a protein from a cell, a cell organelle or from medium.

According to the invention any eukaryotic or prokaryotic cell can be used as host cell for expression or generation of a protein. Particularly preferred examples include fungal cells, animal cells, plant cell, archaebacteria, cyanobacteria, gram-positive bacteria and gram-negative bacteria.

The present invention also relates to a protein obtainable by the inventive process for the production of a protein.

Another aspect of the present invention relates to a process for the production of a fermentation product by cultivating a cell producing the fermentation product and/or at least one enzyme involved in the formation of the fermentation product in a medium wherein the genome of the cell is stabilized against spontaneously occurring sequence changes by at least two mutations whose combined actions lead to an enhanced capability of at least two cellular DNA repair mechanisms to repair spontaneously occurring mutations.

Therefore, the inventive process is directed to the use of a cell for fermentative purposes, i.e. for producing a fermentation product, whereby the genome of the cell is stabilized against spontaneously occurring sequence changes and therefore exhibits very low spontaneous mutation rates. Furthermore, the cell used has an improved cellular viability. By the use of cells with reduced spontaneous mutation rates the inventive process secures that the probability is greatly reduced that a nucleotide sequence encoding a given protein and thus the amino acid sequence of that protein will be changed by mutations. Since these cells exhibit a greatly enhanced cellular viability the use of these cells furthermore secures that no perturbations occur during the fermentation process and that a high productivity of the fermentation product is obtained.

In the context of the present invention the term “fermentation” includes any process for the production of a defined product due to the anaerobic or aerobic metabolism of microbes, fungal cells, plant cells or animal cells or by the use of enzymes from such cells, which can be isolated and/or purified and/or immobilised. “Fermentation” includes all enzymatic-chemical alterations of an organic substrate caused by the action of one or more enzymes of microbial, plant, fungal and/or animal cells. The fermentation product desired can be produced within the cell itself, for example due to the expression of a protein or as a result of the metabolism of the cell, whereby the fermentation product can be accumulated within the cell or can be transported out of the cell into its environment, or the cell produces one or more enzymes which upon secretion outside of the cell catalyse the conversion of a substrate present in the medium to the fermentation produced desired. In case that the fermentation product is a nucleic acid or a protein encoded by a nucleic acid the inventive process is useful for avoiding even minimal contaminations of the fermentation product by mutations and therefore secures a long-term integrity of the fermentation product In case that the generation of the fermentation product is due to the action of one or more enzymes produced by the cell, the inventive process is useful to maintain the integrity of the respective enzymes and thus to maintain the specificity and effectiveness of those metabolic processes leading to the generation of the fermentation product.

The inventive process for the generation of a fermentation product can also be useful to maintain the integrity of gene clusters and/or the gene products encoded by such a gene cluster, which are involved in a pathway, and thus to maintain the specificity and effectiveness of that pathway in which the gene products encoded by such a gene cluster are involved. Examples for such a gene cluster include, without being restricted to, the polyketide synthases (PKSs) cluster. Polyketide metabolites are a large group of natural products generated by bacteria such as actinomycetes and myxobacteria and fungi with diverse structures and biological activities. Complex polyketides are produced by multifunctional PKSs involving a mechanism similar to long-chain fatty acid synthesis in animals. Studies on the erythromycin PKS in Saccharopolyspora erythraea revealed a modular organization. Complex polyketide synthesis follows a processive reaction mechanism, wherein each module within a PKS harbors a string of three to six enzymatic domains that catalyse reactions in nearly linear order.

According to the invention the reduced spontaneous mutation rates and the high cellular viability in the cell used for fermentation purposes are obtained by the use of at least two mutations, the combined actions of which lead to an enhanced capability of at least two cellular DNA repair mechanisms to repair spontaneously occurring mutations in any nucleic acid present within the cell. In particular these at least two mutations enhance the capability of the mismatch repair system, the proof-reading function and/or the SOS repair system to repair spontaneously occurring mutations of the cell. In preferred embodiments of the invention these mutations are selected from a mutation leading to an upregulation of the expression of the MutL protein or a homologous protein thereof, a mutation leading to an upregulation of the expression of the MutS protein or a homologous protein thereof, an antimutator allele of a gene encoding DNA polymerase IV or a homologous protein thereof and an antimutator allele of a gene encoding a subunit of DNA polymerase III or a homologous protein thereof.

In one embodiment of the invention the upregulation of the expression of MutL or a homologous protein thereof and the upregulation of the expression of MutS or a homologous protein thereof, respectively, can be achieved by introducing a vector into that cell used for fermentation, whereby the vector comprises the mutL gene or a gene encoding a homologous protein thereof and the mutS gene or a gene encoding a homologous protein thereof, respectively, under the functional control of one or more regulatory units allowing an overexpression of the respective mut protein in comparison to a corresponding wild-type host cell. Preferably, the vector used for overexpression of either the MutL or MutS protein is a multi-copy plasmid, which comprises the respective mut gene under the functional control of one or more regulatory units. In a preferred embodiment of the invention the regulatory unit controlling the overexpression of the respective mut gene can be an inducible or constitutive promoter.

In another embodiment of the invention the upregulation of the expression of MutL or a homologous protein thereof and the upregulation of the expression of MutS or a homologous protein thereof, respectively, can be achieved by either introduction of one or more additional copies of the respective mut gene into the chromosome(s) of the cell or by one or more mutations to the regulatory units directing the transcription of the native chromosomally located mut gene such that in the cell in comparison to a corresponding wild-type cell a higher production of the respective Mut protein is effected.

In a preferred embodiment of the inventive process the antimutator allele of the gene encoding DNA polymerase IV is dinB10. In another preferred embodiment the antimutator allele of the gene encoding the subunit of DNA polymerase III is dnaE911.

According to the invention the presence of dinB10 and dnaE911 within a cell used for fermentation purposes reduces the spontaneous mutation frequencies of that cell in comparison to a wild-type cell at least 10-fold. The presence of dinB10 and dnaE911 within a cell, which furthermore overexpresses the mutL protein, reduces the spontaneous mutation frequencies in that cell in comparison to a wild-type cell at least 50-fold.

The fermentation products obtained by the inventive process can be primary metabolites or secondary metabolites of the cell. Primary metabolites are those substances which are synthesized by the living cell and which are necessarily required by the cell for the formation of cell structures and for maintaining metabolic processes. In contrast to primary metabolites secondary metabolites are those compounds, in particular low molecular compounds, synthesised by the cell which are not necessarily required for maintenance of the cell's functions. In general secondary metabolites can confer to the cell or organism a selective advantage in a particular environment The fermentation products obtained by the inventive process can be the product of an fermentation in the classical sense, i.e. the enzymatic degradation of carbohydrates with oxygen exclusively effected by microbes, such as the products of an alcoholic fermentation, lactic acid fermentation, propionic acid fermentation etc.

Examples of fermentation products include, without being restricted to, nucleic acids, nucleosides, nucleotides, proteins, amino acids, organic acids, alcohols, carbohydrates, vitamins, antibiotics and alkaloids.

In preferred embodiments of the invention the fermentation products are nucleic acids, in particular such with therapeutic usability, for example nucleic acids which shall be used as vaccine.

In the context of the invention “nucleic acids” are molecules which comprise at least two nucleotides linked by a phosphodiester linkage. The term “nucleic acid” therefore also means an oligonucleotide. A nucleic acid can either be a DNA or a RNA. The nucleic acid can be single-stranded or double-stranded.

In preferred embodiments of the invention the fermentation products are proteins, in particular enzymes or proteins with therapeutic usability, or portions thereof, such as domains. Preferred examples of enzymes. Preferred examples of an, enzyme include, without being restricted to, proteases, amylases, pectinases and glucose-isomerases. Preferred examples of proteins with therapeutic usability include, without being restricted to, cytokines such as interferons, interleukins, growth factors, coagulation factors, antibodies etc.

Preferred examples of organic acids include, without being restricted to, citric acid, lactic acid or acetic acid. Preferred examples of alcohols include, without being restricted to, ethanol, propanol and butanol. Preferred examples of carbohydrates include, without being restricted to, sugars such as sucrose, maltose or palatinose or sugar alcohols such as xylitol or maltitol. Preferred examples of vitamins include, without being restricted to, vitamin B12 or riboflavine. Preferred examples of antibiotics include, without being restricted to, penicillin, cephalosporin, streptomycin, a polyketide antibiotic such as erythromycin etc.

The fermentation product can be isolated either from the fermenting cells or from the medium used for cultivation.

Therefore, the present invention also relates to a fermentation product obtainable by the inventive process for the production of a fermentation product.

In the inventive process or the generation of a fermentation product both eukaryotic and prokaryotic cells can be used. In a preferred embodiment of the inventive process for the production of a fermentation product the eukaryotic cell used is a fungal cell, an animal cell or a plant cell. Preferably, the prokaryotic cell used in the inventive process for the production of a fermentation product is a cyanobacterium, archaebacterium, a gram-positive bacterium or a gram-negative bacterium.

Of course it is possible to use any cell in the present process for the generation of a fermentation process, that has in comparison to a corresponding wild-type cell a, preferably greatly, reduced spontaneous mutation rate. In particular the use of that cells or organisms is preferred, whose spontaneous mutation frequencies have been reduced by the inventive process for reducing spontaneous mutation rates or which have been generated by the inventive process for generation of a cell or organism with reduced spontaneous mutation frequencies.

In a preferred embodiment of the inventive process the cell is cultivated in a suitable liquid medium. The artisan knows numerous, liquid media in which cells, for example prokaryotic cells or eukaryotic cells, can be cultivated in order to produce a fermentation product. According to the invention the cell can be cultivated either in a continuous culture or in a batch culture. A batch culture is a culture, which starts from a liquid medium seeded with cells, whereby during the cultivation the medium is not continuously exchanged and optionally only a gas such as oxygen is introduced. The batch culture can be a fed batch culture in order to circumvent a catabolic repression of the product formation. In case of a fed batch culture therefore the consumption of substrate such as a given sugar will be compensated by the delivery of additional amounts of that substrate. A continuous culture is a culture, wherein the culture is only once inoculated with cells and afterwards depending on the increase in cell mass or accumulation of the product the culture medium consumed is continuously replaced with fresh medium, whereby the fermentation product together with medium consumed and optionally cells are simultaneously removed from the culture. Continuous cultures can be carried out in fermenting tanks.

In a preferred embodiment of the invention the cells can be present in the culture in immobilised form. In another embodiment of the inventive process the cell is cultivated on a solid or semi-solid medium.

In a preferred embodiment the present invention relates to an organism, in particular a prokaryotic organism, most preferably an Escherichia coli strain, which is selected from the group consisting of:

E. coli MG1655dinB10 containing plasmid pmutL (DSM 17016),

E. coli MG1655dinB10 mutL::tet containing plasmid pmutL (DSM 17017),

E. coli MG1655 dnaE zae::cm containing plasmid pmutL (DSM 17018),

E. coli MG1655 dnaE zae::cm mutL::tet containing plasmid pmutL (DSM 17019),

E. coli MG1655dinB10 dnaE zae::cm (DSM 17015),

E. coli MG1655dinB10 dnaE zae::cm mutL::tet (DSM 17014),

E. coli MG1655dinB10 dnaE zae::cm containing plasmid pmutL (DSM 17020) and

E. coli MG1655dinB10 dnaE zae::cm mutL::tet containing plasmid pmutL (DSM 17021).

The above-identified microorganisms have been deposited according to the Budapest Treaty with the DSMZ (Deutsche Sammlung für Mikroorganismen und Zelikulturen GmbH in Braunschweig, Germany) on the 3 Jan. 2005 under the above-identified DSM-numbers.

The present invention is illustrated by the following sequence listing, figures and examples.

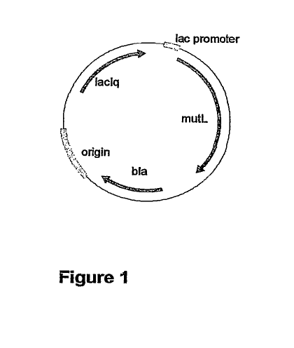

FIG. 1 shows the physical structure of plasmid pmutL, which has in principal the same structure as plasmid pmutS.

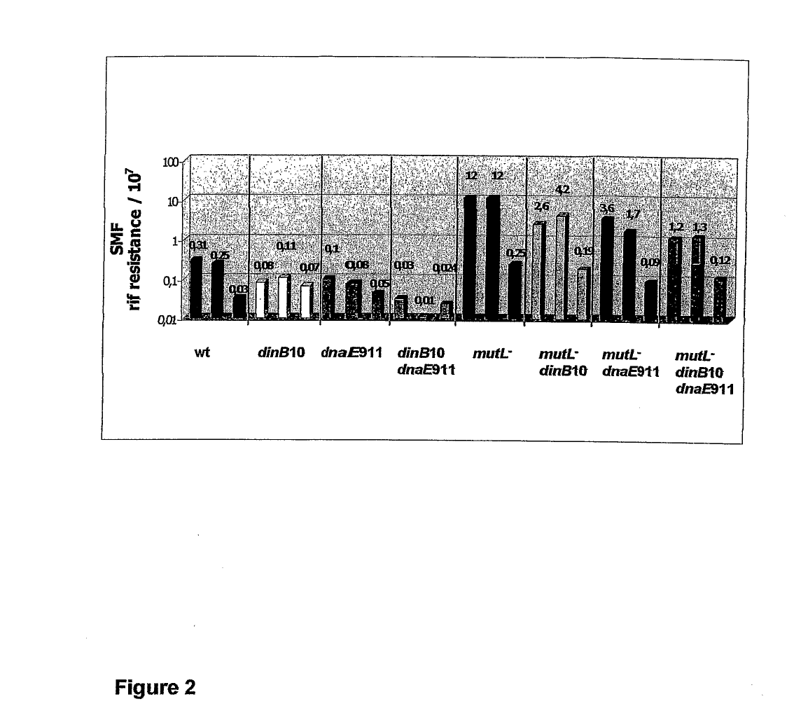

FIG. 2 is a graphical representation of the values of spontaneous mutation frequencies to rifampicin resistance in wild-type, dinB10, dnaE911, dinB10 dnaE911, mutL⁻, mutL⁻ dinB10, mutL⁻ dnaE911 and mutL⁻ dinB10 dnaE911 strains. As the figure shows, the mutability is strongly suppressed by the expression of MutL. Data are presented as SMF values. Plasmid pmutL is a mutL⁺ derivative of plasmid pTrcHis2/lacZ. Wild-type strain MG1655 (wt) was chosen for the whole set of genetic manipulations. Relevant genotypes are indicated. Columns are divided into triplets, wherein each triplet shows the same genotype and is composed of a) first column: bacterial strain, b) corresponding bacterial strain carrying plasmid pTrcHis2/lacZ, and c) corresponding bacterial strain carrying plasmid pmutL.

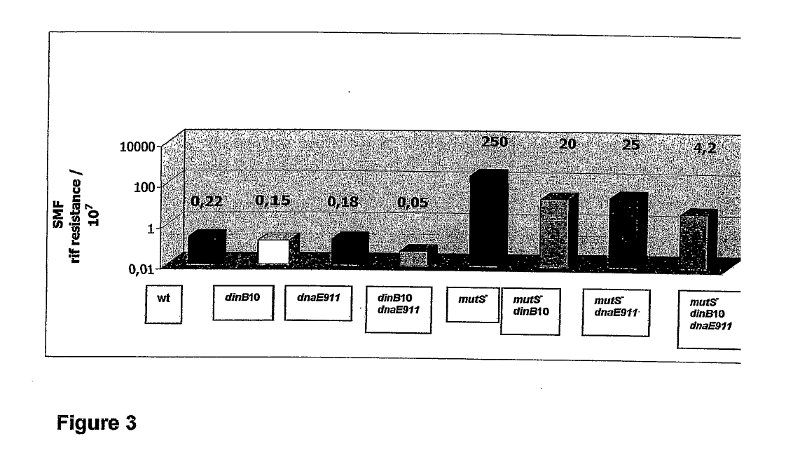

FIG. 3 is a graphical representation of the values of spontaneous mutation frequencies to rifampicin resistance in wild-type, dinB10, dnaE911, dinB10 dnaE911, mutS⁻, mutS⁻ dinB10, mutS⁻ dnaE911 and mutS⁻ dinB10 dnaE911 strains. The figure shows the suppression of the mutability in a mutS⁻ background by dinB10, dnaE911 and dinB10 dnaE911. Data are presented as SMF values. Relevant genotypes are indicated.

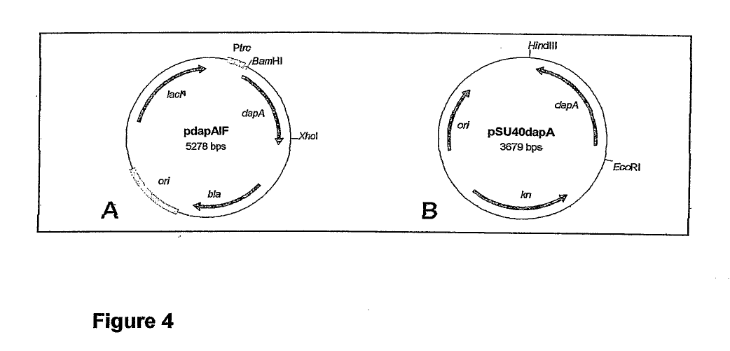

FIG. 4 shows the physical structure of plasmid pdapAIF (A) and plasmid pSU40dapA (B).

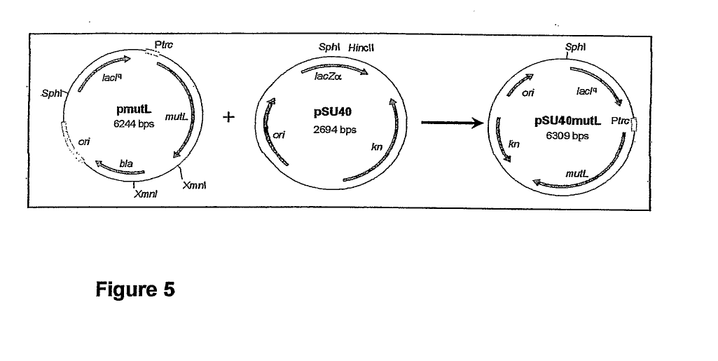

FIG. 5 shows the strategy for generating plasmid pSU40mutL.

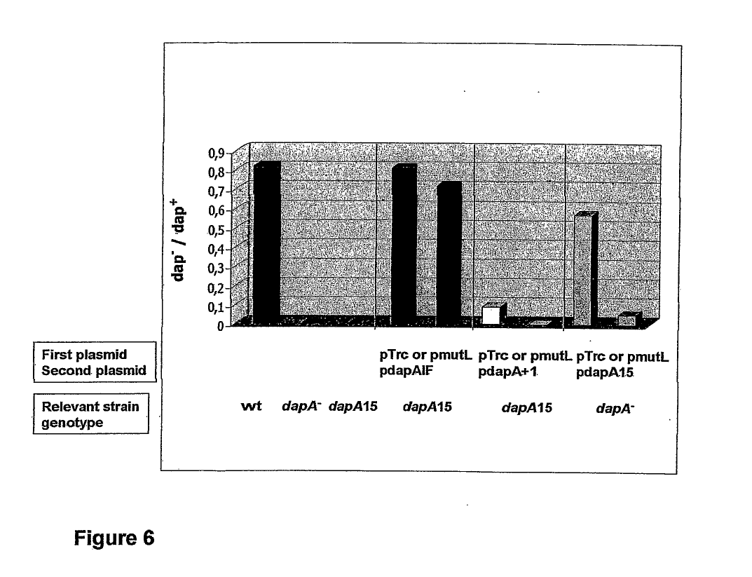

FIG. 6 is a graphical representation of the reversion of dapA point mutations in strains Top10 (wild-type; dapA⁺), MG1655 dinB10 dapA::kn (dapA⁻), AT997 (dapA15, dapA⁻), AT997 pdapAIF pTrc (dapA⁺), AT997 pdapAIF pmutL (dapA⁺), AT997 pdapA+1 pTrc (dapA⁻), AT997 pdapA+1 pmutL (dapA⁻), MG1655 dinB10 dapA::kn pdapA15 pSU40 (dapA⁻), MG1655 dinB10 dapA::kn pdapA15 pSU40mutL (dapA⁻). The figure shows the suppression of the mutability by the expression of MutL. The values of mutations are presented that confer resistance to DAP divided by the total number of viable cells. Plasmid pmutL is a mutL⁺ derivative of plasmid pTrcHis2/lacZ or pSU40. Wild-type strain MG1655 (wt) was chosen for the whole set of genetic manipulations. Relevant genotypes are indicated. The figure is divided into a triplet of controls and three duplets of strains investigated. Each duo is composed of a) first column: bacterial strain carrying a parental vector and b) bacterial strain carrying the mutL⁺ derivative. All bacterial strains used are expressing chromosomal wild-type MutL.

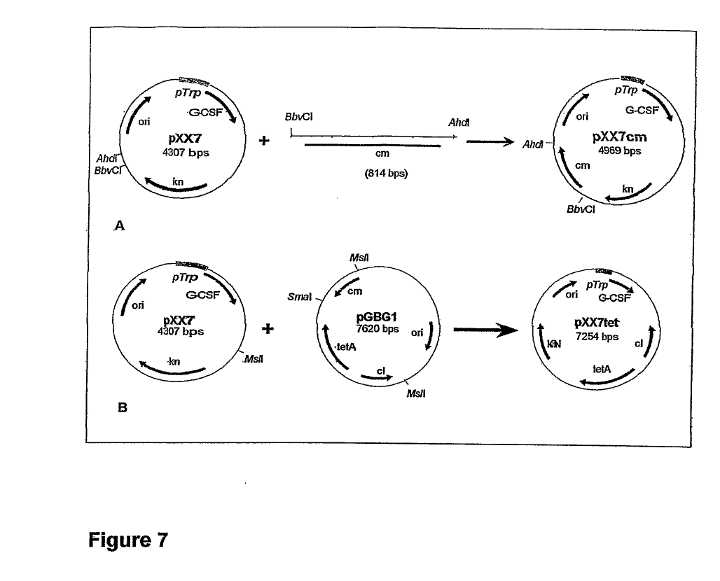

FIG. 7 shows derivatives of plasmid pXX7.
A) Cloning of a chloramphenicol cassette into pXX7 whereby plasmid pXX7cm was obtained. Nine of the obtained clones were analysed by restriction digestion with EcoRI and NcoI (expected fragments: 1677 bps and 3292 bps). All of the tested clones showed the correct migration pattern.
B) Cloning of a silent tetA gene into plasmid pXX7 whereby plasmid pXX7tet was obtained. Twenty-four of the obtained clones were analysed by restriction digestion with EcoRI (expected fragments: 4141 bps and 3113 bps). One of the tested clones showed the correct migration pattern.

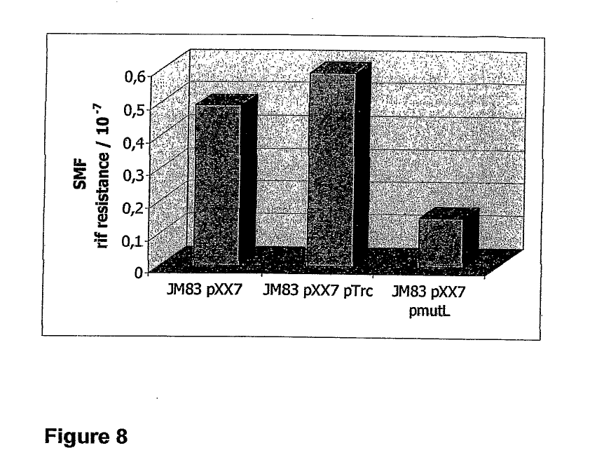

FIG. 8 shows spontaneous mutation frequencies to rifampicin resistance of strains JM83 pXX7, JM83 pXX7 pTrc and JM83 pXX7 pmutL. Plasmid pmutL is a mutL⁺ derivative of plasmid pTrc. SMF values are given of rifampicin resistant versus total number of viable cells. Wild-type strain JM83 expresses chromosomal wild-type MutL.

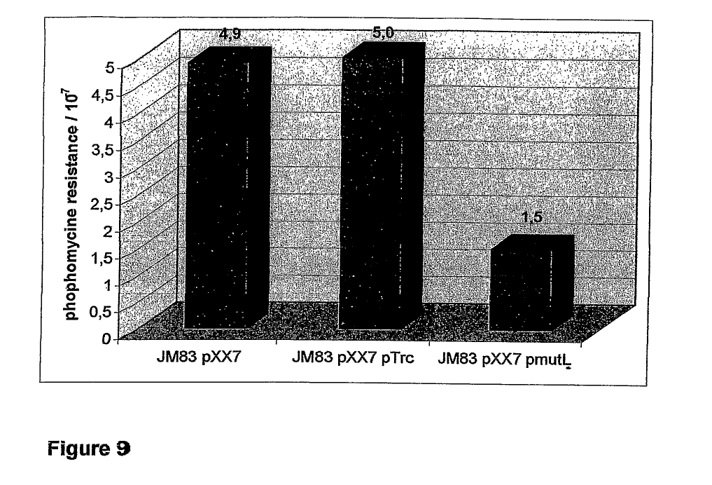

FIG. 9 shows the values for spontaneous mutation frequencies to phosphomycine resistance of strains JM83 pXX7, JM83 pXX7 pTrc and JM83 pXX7 pmutL. Plasmid pmutL is a mutL⁺ derivative of plasmid pTrc. SMF values are given of phosphomycin resistant versus total number of viable cells. Wild-type strain JM83 expresses chromosomal wild-type MutL.

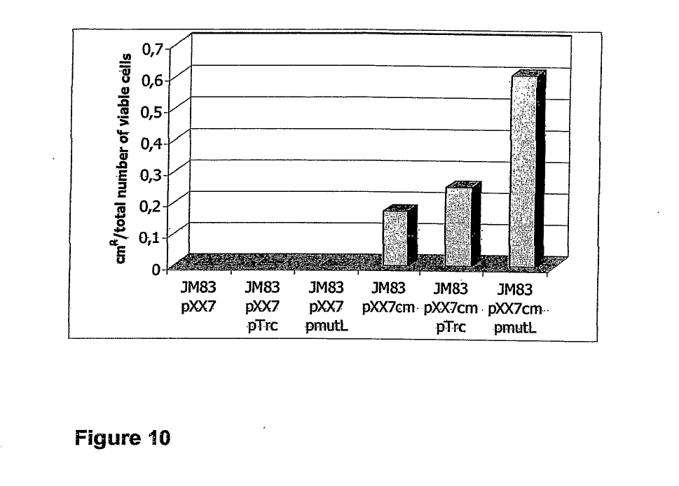

FIG. 10 shows the number of cm^{R} colonies due to spontaneous mutation frequencies in the chloramphenicol resistance reporter gene for strains JM83 pXX7cm, JM83 pXX7cm pTrc and JM83 pXX7cm pmutL. Plasmid pXX7cm is the cm⁺ derivative of pXX7. Plasmid pmutL is a mutL⁺ derivative of pTrc. Presented are the values of mutations that introduce sensitivity to chloramphenicol divided by the total number of viable cells. Control strains JM83pXX7, JM83pXX7 pTrc and JM83pXX7 pmutL show the expected sensitivity to chloramphenicol (carrying no chloramphenicol resistance gene).

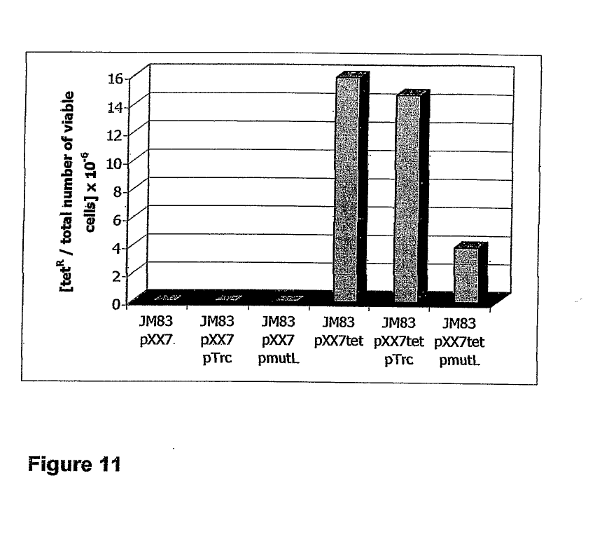

FIG. 11 shows the number of tet^{R} colonies due to spontaneous mutation frequencies in the silent tetA reporter gene for strains JM83 pXX7tet, JM83 pXX7tet pTrc and JM83 pXX7tet pmutL. Plasmid pXX7tet is a derivative of pXX7 carrying a silent tetA. Plasmid pmutL is a mutL⁺ derivative of pTrc. Presented are the values of mutations that introduce resistance to tetracycline divided by the total number of viable cells. Control strains JM83pXX7, JM83pXX7 pTrc and JM83pXX7 pmutL show the expected sensitivity to tetracycline (carrying no tetracycline resistance gene). For the two strains JM83 pXX7 and JM83 pXX7tet the mutability values were 0.43 or 0.39 (Δ1.1). Expression of MutL drops the value of mutability to 0.25 (Δ1.72).

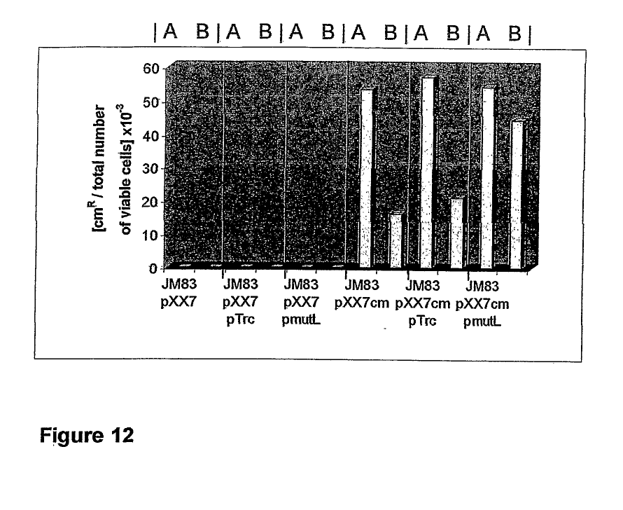

FIG. 12 shows the mutability of the modified producer strain JM83 pXX7cm during “fermentation conditions”. As shown the frequency of chloramphenicol resistance of the cells when G-CSF is suppressed (A) or expressed (B). In each tested case, MutL was chromosomally expressed. Control strains JM83 pXX7, JM83 pXX7pTrc and JM83 pXX7pTrc pmutL show the expected sensitivity to chloramphenicol in case (A) and (B).

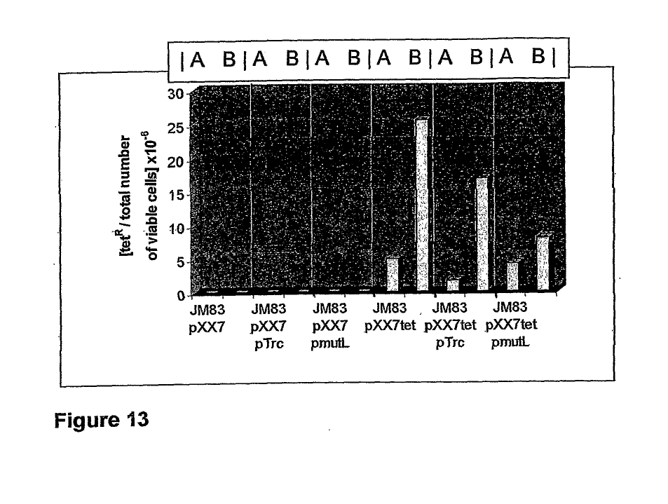

FIG. 13 shows the mutability of the modified producer strain JM83 pXX7tet during “fermentation conditions”. The figure shows the frequency of tetracycline resistance of the cells when G-CSF is suppressed (A) or expressed (B). In each tested case, MutL was chromosomally expressed.

SEQ ID No. 1 and 2 show the sequences of the primers MutLNcoIhin and MutLXhoIher, respectively, for the amplification of a DNA fragment encoding the MutL protein.

SEQ ID No. 3 and 4 show the sequences of the primers MutSBamHIhin and MutSXhoIher, respectively, for the amplification of a DNA fragment encoding the MutS protein.

SEQ ID No. 5 to SEQ ID No. 10 show the sequence of the primers dapABamHIIF, dapABamH1+1, dapABamH1+2, dapAXhoI, dapAEcoRIhin and dapAHindIIIher, respectively, for the amplification of a DNA fragment encoding the DapA protein.

SEQ ID No. 11 and 12 show the sequences of the primers cmBbvCIhin and cmAhdIher, respectively, for the amplification of a DNA fragment encoding chloramphenicol resistance.

### EXAMPLE I

Determination of Spontaneous Mutation Frequencies (SMF) to Resistance to Rifampicin in Different E. coli Strains

Cells of the bacteria Escherichia coli are normally sensitive to the antibiotic rifampicin; that is, they are unable to grow on media that contains the antibiotic. However, mutations that confer resistance to rifampicin naturally occur at a low frequency in the genome of E. coli. Thus, if one plates a large number of cells (10⁸) on media that contains rifampicin, a few colonies will grow whereas most cells will be killed by the antibiotic. This is possible because mutations that confer resistance to rifampicin have spontaneously occurred in the genome of the cells. The mutations are, in turn, inherited by progeny of the original mutant. It is important to note that this mutation is only beneficial in an environment wherein rifampicin is present, and it probably confers no advantage to the variant under most other conditions. Rifampicin is an antibiotic mostly used to treat tuberculosis and leprosy, and, occasionally, other diseases. Resistance is due to alterations in membrane permeability or to mutation in the rpoB gene coding for mRNA polymerase. Both these mechanisms originate via chromosomal mutation. However, there exist several target genes within the chromosome of E. coli, which can confer bacterial resistance against rifampicin.

The frequency of spontaneous mutations that confer resistance to rifampicin divided by the total number of viable cells in the culture was determined to calculate the spontaneous mutation frequency (SMF).

$$SMF = \frac{Number of {rif}^{ R} mutants}{Total number of viable cells}$$

A) Overexpression of MutL in an E. coli Strain that Exhibits a Strong Mutator Phenotpype (Chromosomal Deletion of mutL and/or dnaQ) and Plating Out the Cells on the Antibiotic Rifampicin.
Experimental Outline

1. Construction of MutL and MutS overexpression plasmid

a) Cloning mutL in pTrcHis2/lacZ

This plasmid was constructed to achieve high and “controlled” expression of MutL in E. coli.

Genomic DNA prepared from E. coli strain MG1655 was used to amplify an 1848 bp DNA fragment encoding the MutL protein by primers 1 (SEQ ID No. 1) and 2 (SEQ ID No. 2) (see also table 1). This PCR fragment was digested and cloned into the plasmid pTrcHis2/lacZ as a NcoI-XhoI fragment to yield plasmid pmutL. The physical structure of plasmid pmutL is shown in FIG. 1. PCR conditions were as follows (temperature in ° C./time in min): (98/10) (96/0.75; 55/0.50; 72/2.5)₃₅ (72/10); relation (Taq/Pfu) (5/1).

Six of the obtained clones were isolated and analysed by restriction digestion with EcoRV (expected fragments: 871 bps and 5373 bps). All six clones showed the correct migration pattern.

b) Cloning mutS in pTrcHis2/lacZ

This plasmid was constructed to achieve a high and “controlled” expression of MutS in E. coli.

Genomic DNA prepared from E. coli strain MG1655 was used to amplify a 2562 bp DNA fragment encoding for the MutS protein by primers 3 (SEQ ID No. 3) and 4 (SEQ ID No. 4) (see table 1). This PCR fragment was digested and cloned into the plasmid pTrcHis2/lacZ as a BamHI-XhoI fragment to yield plasmid pmutS. Plasmid pmutS has in principal the same physical structure as plasmid pmutL shown in FIG. 1.

PCR conditions were as follows (temperature in ° C./time in min): (98/10) (96/0.75; 55/0.58; 72/3)₃₅ (72/10); Herculase.

Six of the obtained clones were isolated and analysed by restriction digestion with EcoRV (expected fragments: 1186 bps and 5775 bps). All six clones showed the correct migration pattern.

TABLE 1

List of primers used for amplification of
DNA fragments encoding the MutL and the
MutS protein, respectively.

| | |
|---|---|
| Primer | Sequence |

| | | |
|---|---|---|
| 1 | MutLNcol- | CATGCCATGGCGCCAATTCAGGTCTTACCGCCAC |
| | hin | AAC |
| 2 | MutLXhol- | CCGCTCGAGCCTCATCTTTCAGGGCTTTTATCGC |
| | her | CGG |
| 3 | MutS- | CGCGGATCCGAGTGCAATAGAAAATTTCGACGCC |
| | BamHIhin | CATA |
| 4 | MutSXhol- | CCGCTCGAGCCACCAGGCTCTTCAAGCGATAAAT |
| | her | CCAC |

2. Western Blot Analysis
a) Detection of MutL

20 μl overnight culture of ES568 (mutL13; Mut⁻) pmutL was inoculated in 10 ml LB containing 0.4% glucose and ampicillin (100 μg/m) until an OD of approximately 0.5 was reached. To remove the glucose the cells were centrifuged and resolved in 5 ml LB containing the required antibiotics. Adding IPTG at a final concentration of 1 mM induced expression from P_{lac} of pmutL. The cultures were incubated 16 hours at 37° C. than centrifuged and prepared for SDS-PAGE analysis. Western Blot analysis was done using AP conjugated anti-His-tac antibodies (data not shown).

b) Detection of MutS

Wild-type cells of strain Top10 were transformed with the constructed plasmid pmutS and the obtained transformants were purified. 20 μl overnight cultures of these bacteria were inoculated in 10 ml LB containing ampicillin (100 μg/ml) and 0.4% glucose until an OD of approximately 0.5 was reached. To remove the glucose the cells were centrifuged and resolved in 5 ml LB containing the required antibiotics. Adding IPTG at a final concentration of 1 mM induced expression from P_{lac} of pmutS. The cultures were incubated 16 hours at 37° C., than centrifuged and prepared for SDS-PAGE analysis. Western blot analysis was done using AP conjugated anti-His-tac antibodies (data not shown).

3. Growth Studies

Bacterial strains ES568 (mutL13; Mut⁻), MG1655dnaQ::kn (Mut⁻) and wild-type strain MG1655 (Mut⁺) were transformed with plasmid pmutL or the parental plasmid pTrcHis2/lacZ. Obtained single colonies were purified on LB agar plates containing ampicillin 100 μg/ml and then inoculated in LB containing ampicillin 100 μg/ml overnight at 37° C.

20 μl overnight cultures of these bacteria were inoculated in 5 ml LB containing 0.4% glucose and the required antibiotic (ampicillin 100 μg/ml; kanamycin 50 μg/ml) until an OD of approximately 0.5 was reached. To remove the glucose the cells were centrifuged and resolved in 5 ml LB containing the corresponding antibiotics. Adding IPTG at a final concentration of 1 mM induced expression from P_{lac} of pmutL. The cultures were incubated overnight at 37° C., the cells were centrifuged and the media volume reduced to 1/10 (v/v).

Finally, cultures were diluted and plated out on agar plates containing either 100 μg/ml ampicillin or 100 μg/ml ampicillin plus 100 μg/ml rifampicin and incubated for three days at 30° C.

Expression of MutL was confirmed by Western Blot analysis.

Summary and Conclusions: It was observed that overexpression of MutL (in the mutL⁺ background) reduces the frequency of spontaneous mutations by a factor of about 20. A similar effect was observed for mutL⁻ or dnaQ⁻ cells, which exhibit a high mutation rate (data not shown).

B) Overexpression of MutL in an E. coli Strains Carrying Different Combinations of dinB Deletion and/or the dnaE911 Antimutator Allele.

1. Strain Construction

a) P1ᵥᵢᵣ lysate was prepared from E. coli strain ES1484 (mutL218::Tn1:0) to infect bacterial cells from the E. coli strains MG1655 and MG1655dinB10.

Preparation of P1ᵥᵢᵣ

An aliquot of an overnight culture from the E. coli strain ES1484 (mutL218::tet) was inoculated in LB containing CaCl₂ (5 mM) at 37° C. During the exponentially growing phase, P1ᵥᵢᵣ (of the wild-type strain MG1655) was added. The infected culture was incubated for ca. 4 h at 37° C. until cell lysis was observed. CHCl₃ was added, the lysate centrifuged, the supernatant transferred to a new tube containing CHCl₃ and stored at 4° C.

P1-Transduction:

Aliquots of overnight cultures from the host strains MG1655 and MG1655 dinB10 were inoculated in LB at 37° C. until the exponentially growing phase was reached. Cells were centrifuged, resuspended in MgSO₄ (c_{E}=1.0 mM) containing CaCl₂ (c_{E}=5 mM), the prepared P1ᵥᵢᵣ lysate added and the cell mixtures incubated for 30 min at RT. Na-citrate was added (to stop the phage infection) and SOC as source of nutrients. After incubation for 1 h at 37° C. cell aliquots were plated out on agar plates containing tetracycline (12.5 μg/ml). Obtained single colonies were purified, tested and isolated strains stocked.

b) P1ᵥᵢᵣ lysates were prepared from dnaE strain NR10171 (dnaE911 zae::cm) to infect bacteria cells from the E. coli strains MG1655, MGG1655dinB10, MG1655mutL::tet and MG1655dinB10 mutL::tet. Obtained single colonies were purified, tested and isolated strains stocked.

2. Growth Studies

Bacterial strains MG1655, MG1655dinB10, MG1655dnaE911 zae::cm, MG1655dinB10 dnaE911 zae::cm, MG1655mutL::tet, MG1655dinB10 mutL::tet, MG1655dinB10 dnaE911 zae::cmmutL::tet and MG1655dinB10 dnaE911 zae::cm mutL::tet were transformed with plasmids pTrcHis2A or pmutL. Obtained single colonies were purified and inoculated in LB containing 100 μ/ml ampicillin overnight at 30° C.

20 μl of overnight cultures of these bacteria were inoculated in 5 ml LB containing 100 μg/ml ampicillin at 30° C. During the exponentially growing phase, IPTG was added at a final concentration of 1 mM to enhance the expression of MutL from P_{lac}. The cultures were kept 1 hour at 30° C., then the temperature was shifted to 37° C. and the cells were incubated overnight at 37° C.

The cells were centrifuged and the volume of the media reduced to 1/10 (v/v).

Finally, cultures were diluted and plated out on agar plates containing 100 μg/ml ampicillin or 100 μg/ml ampicillin plus 100 μg/ml rifampicin and incubated for three days at 30° C.

The values for spontaneous mutation frequencies (SMF) to resistance to rifampicin are presented in table 2 and FIG. 2 showing the suppression of mutability by the expression of MutL.

TABLE 2
SMF values of rif resistant colonies

| | | SMF (rif resis- |
|---|---|---|
| Strain | Plasmid | tance/10⁻⁷) |
| MG1655 | | 0.31 |
| MG1655 | pTrcHis2A | 0.25 |
| MG1655 | pmutL | 0.034 |
| MG1655dinB10 | | 0.083 |
| MG1655dinB10 | pTrcHis2A | 0.11 |
| MG1655dinB10 | pmutL | 0.068 |
| MG1655 dnaE zae::cm | | 0.10 |
| MG1655 dnaE zae::cm | pTrcHis2A | 0.08 |
| MG1655 dnaE zae::cm | pmutL | 0.045 |
| MG1655dinB10 dnaE | | 0.032 |
| zae::cm | | |
| MG1655dinB10 dnaE | pTrcHis2A | 0.011 |
| zae::cm | | |
| MG1655dinB10 dnaE | pmutL | 0.024 |
| zae::cm | | |
| MG1655 mutL::tet | | 11.86 |
| MG1655 mutL::tet | pTrcHis2A | 11.8 |
| MG1655 mutL::tet | pmutL | 0.25 |
| MG1655dinB10 mutL::tet | | 2.56 |
| MG1655dinB10 mutL::tet | pTrcHis2A | 4.20 |
| MG1655dinB10 mutL::tet | pmutL | 0.19 |
| MG1655 dnaE zae::cm | | 3.59 |
| mutL::tet | | |
| MG1655 dnaE zae::cm | pTrcHis2A | 1.74 |
| mutL::tet | | |
| MG1655 dnaE zae::cm | pmutL | 0.093 |
| mutL::tet | | |
| MG1655dinB10 dnaE | | 1.18 |
| zae::cm mutL::tet | | |
| MG1655dinB10 dnaE | pTrcHis2A | 1.31 |
| zae::cm mutL::tet | | |
| MG1655dinB10 dnaE | pmutL | 0.12 |
| zae::cm mutL::tet | | |

Mutability (SMF values) decreases via the action of antimutators like dinB10 (deletion) or dnaE911 (allele) by a factor of 4 and 3, respectively. Furthermore, an additional reduction of the mutability was observed by the expression of MutL in those strains (factor 2 to 7). A bacterial strain carrying both antimutators, dinB10 and dnaE911, shows a 10 times reduced mutability in comparison to the wild type strain MG1655. Additional expression of MutL does not seem to change the mutability.

Chromosomal deletion of the mutL gene increases drastically the mutability of the host strain about a factor of 10³. This mutability was reduced via antimutators dinB10 (factor 5), dnaE911 (factor 3), dinB10 and dnaE911 (factor 10). Additional induction of MutL drops the mutability up to a factor of 50 (for MG1655 mutL::tet: factor of 50; for MG1655dinB10 mutL::tet: factor of 22; for MG1655 dnaE911 zae::cm; mutL::tet: factor of 20 and for MG1655dinB10 dnaE911 zae::cm mutL::tet: factor of 11).

Summary and Conclusion: The antimutators dinB10 and dnaE911 reduce the spontaneous mutation frequency 3- to 4-fold. The expression of MutL in these strains further reduced this frequency 2- to 7-fold.

C) Mutability in a mutS⁻ Strain Carrying Different Combinations of dinB Deletion and/or the dnaE911 Antimutator

To confirm the results mentioned above, the antimutator effect of dinB10 (deletion of polymerase IV) and/or dnaE911 (allele within the coding sequence for the α-subunit of polymerase III) was tested in a mutS⁻ background.

1. Strain Constructions

P1ᵥᵢᵣ lysate was prepared from E. coli strain ES1481 (mutL215::Tn10) to infect bacterial cells from the E. coli strains MG1655, MG1655dinB10, MG1655dnaE911 zae::cm and MG1655dinB10 dnaE911 zae::cm. Obtained single colonies were purified, tested and the strains stocked.

2. Growth Studies

10 μl of overnight cultures of these bacteria were inoculated in 10 ml LB containing 12.5 μg/ml, tetracycline at 30° C. During the exponentially growing phase (after 8 h growth), the temperature was shifted to 37° C. and the cells were incubated overnight at 37° C.

The cells were centrifuged and the media volume reduced to 1/10 (v/v).

Finally, cultures were diluted and plated out on agar plates with and without rifampicin (100 μg/ml) and incubated for five days at 30° C.

The values for spontaneous mutation frequencies (SMF) to resistance to rifampicin are presented in table 3 and FIG. 3 showing the suppression of mutability in a mutS⁻ background.

TABLE 3
SMF values of rif resistant colonies

| | | SMF (rif resis- |
|---|---|---|
| Strain | Plasmid | tance/10⁻⁷) |
| MG1655 | — | 0.218 |
| MG1655dinB10 | — | 0.145 |
| MG1655 dnaE911 zae::cm | — | 0.18 |
| MG1655dinB10 dnaE911 | — | 0.048 |
| zae::cm | | |
| MG1655 mutS::tet | — | 252.67 |
| MG1655dinB10 mutS::tet | — | 20.16 |
| MG1655 dnaE911 zae::cm | — | 24.84 |
| mutS::tet | | |
| MG1655dinB10 dnaE | — | 4.24 |
| zae::cm mutS::tet | | |

Mutability (SMF values) decreases in the presence of antimutators like dinB10 or dnaE911 (about a factor 1.5) in comparison to the wild-type. Furthermore, a bacterial strain carrying the antimutators dinB10 and dnaE911 shows a 4.5 fold reduced mutability in comparison to the wild-type MG1655.

Chromosomal deletion of the mutS gene increases dramatically the mutability in the host strain. This mutability was reduced by antimutators dinB10 (factor 12.5), dnaE911 (factor 10), dinB10 and dnaE911 (factor 60).

Conclusion and summary: The antimutator effect caused by dinB10 or dnaE911 complements partially the Mut⁻ phenotype caused by the chromosomal deletion of mutS.

### EXAMPLE II

Reversion of dapA Mutations

In order to study the reversion of point and frameshift mutations in a particularly target gene a system was used that possesses an auxotroph phenotype in E. coli (dapA⁻ strain).

The dapA gene encodes the dihydrodipicolinate synthase which catalyses the condensation of L-aspartate-β-semialdehyde and pyruvate to dihydropicolinic acid via a ping-pong mechanism in which pyruvate binds to the enzyme by forming a Schiff-base with a lysine residue. Bacterial cells with a mutation in the dapA gene that leads to an unfunctional gene product are unable to grow on media that does not contain DL-diaminopimelic acid (DAP).

MutL is expressed in a dapA⁻ background (dapA15 (point mutation possesses DapA⁻ phenotype) or dapA::kn) in the presence of plasmids encoding dapAIF (the wt ORF), dapA+1 (a frameshift mutation), dapA+2, or dapA15 (point mutation). Revertants are detected on plates lacking DL-diaminopimelic acid.

Experimental Outline

1. Construction of Plasmids

a) Cloning of dapA

Cloning dapA (dapA15), dapA+1 (dapA15+1), dapA+2 (dapA15+2) into pTrcHis2/lacZ

Genomic DNA prepared from E. coli strain MG1655 (wt) or AT997 (dapA15) was used to amplify a 732 bp DNA fragment encoding for the DapA protein by primers 5 (SEQ ID No. 5), 6 (SEQ ID No. 6), 7 (SEQ ID No. 7) and 8 (SEQ ID No. 8) (see table 4). These PCR fragments were digested and cloned into the plasmid pTrcHis2/lacZ as a BamHI-XhoI fragment to yield plasmids pdapAIF, pdapA+1 and pdapA+2. The physical structure of plasmid pdapAIF is shown in FIG. 4 A. PCR conditions were as follows (temperature in ° C./time in min): (98/10) (96/0.75; 55/0.50; 72/2)₃₅ (72/10); Herculase.

In each case eight of the obtained clones were analysed by restriction digestion with BstEII (expected fragments: 1314 bps and 3964 bps). Collectively twenty-three of the twenty-four tested clones showed the correct migration pattern.

Cloning dapA15 into pSU19 and pSU40

Prepared genomic DNA from E. coli strain AT997 was used to amplify a 732 bp DNA fragment encoding for the DapA protein by primers 9 (SEQ ID No. 9) and 10 (SEQ ID No. 10) (see table 4). This PCR fragment was digested and cloned into plasmids pSU19 and pSU40 as a HindIII-EcoRI fragment to yield plasmids pSU19dapA15 and pSU40dapA15. The physical structure of plasmid pSU40dapA is shown in FIG. 4B.

PCR conditions were as follows (temperature in ° C./time in min) (98/10) (96/0.75; 55/0:50; 72/2)₃₅ (72/10); relation (Taq/Pfu) (5/1).

Ten of the observed clones were isolated and analysed by restriction digestion with NheI (expected fragments: 1122 bps and 2557 bps for pSU40) or ApoI (expected fragments: 478 bps and 3192 bps). All clones showed the correct migration pattern.

2) Cloning of mutL

a) Subcloning of mutL into pSU40

Plasmid pmutL was SpHI-XmnI digested and a 3629 bp fragment isolated by gel electrophoresis. Plasmid pSU40 was SphI-HincII digested and a 2680 bp fragment isolated by gel electrophoresis. The two purified fragment were sticky-plank ligated to yield in plasmid pSU40mutL. The strategy for the generation of plasmid pSU40mutL is shown in FIG. 5.

Six of the obtained clones were analysed by restriction digestion with NcoI (expected fragments: 2675 bps and 3634 bps), PvuI (expected fragments: 1368 bps and 4941 bps) and BglI (expected fragments: 1828 bps and 4481 bps). Three of the six tested clones showed the correct migration pattern.

b) Plasmid pmutLspec: Substitution of bla by spec

Substitution of the ampicillin resistance gene by a spectomycin resistance gene in plasmid pmutL.

Prepared DNA from plasmid pIC156 was FspI-XmnI digested and a 1304 bp fragment isolated using gel electrophoresis, and prepared DNA from plasmid pmutL was ScaI-XmnI digested and a 5443 bp fragment isolated using gel electrophoresis. The two purified fragments were blank-blank ligated to yield plasmid mutLspec.

Twelve of the obtained clones were analysed by restriction digestion with EcoRI (expected fragments: 1291 bps and 5456 bps). Two of the twelve tested clones showed the correct size.

TABLE 4

List of primers used for amplification of
DNA fragments encoding the DapA protein

| | |
|---|---|
| Primer | Sequence |

| | | |
|---|---|---|
| 5 | DapABamHIIF | CGCGGATCCGTTCACGGGAAGTATTGTCG |
| | | CGATTG |
| 6 | dapABamHI+1 | CGCGGATCCGCTTCACGGGAAGTATTGTC |
| | | GCGATTG |
| 7 | dapABamHI+2 | CGCGGATCCGCGTTCACGGGAAGTATTGT |
| | | CGCGATTG |
| 8 | DapAXhol | CCGCTCGAGCCAGCAAACCGGCATGCTTA |
| | | AGCGCC |
| 9 | DapAEcoRIhin | CCGGAATTCCGGGCATACAACAATCAGAA |
| | | CGGTTCTGTC |
| 10 | dapAHindIIIher | CCCAAGCTTGGGCGAAACACCCGCAACCT |
| | | TTGCCAGGCG |

2. Western Blot Analysis
a) Detection of DapA/DapA15

Strain dapA::kn was transformed with pdapAIF, pdapA+1, pdapA+2, pdapA15IF, pdapA15+1 and pdapA15+2 and the observed transformants were purified.

20 μl overnight cultures of these bacteria were inoculated in 10 ml LB containing 0.4% glucose, DL-diaminopimelic acid (100 μg/ml), ampicillin (100 μg/ml) and kanamycin (50 μg/ml) until an OD of approximately 0.5 was reached. To remove the glucose one part of the cells were centrifuged and resolved in 5 ml LB containing the required antibiotics. Adding IPTG at a final concentration of 1 mM induced expression from P_{lac} of the different pTrcHis2/lacZ derivatives. The cultures were incubated for 16 hours a 37° C., then centrifuged and prepared for SDS-PAGE analysis. Western Blot analysis was done using AP conjugated anti-His-tac antibodies (data not shown).

b) Detection of MutL

Wild-type strain Top10 was transformed with the plasmid pmutLspec and the obtained transformants were purified 20 μl overnight cultures of these bacteria were inoculated in 10 ml LB containing spectinomycin (75 μg/ml) until an OD of approximately 0.5 was reached. The culture was divided into 5 ml aliquotes and once IPTG was added at a final concentration of 1 mM to induce expression of MutL from the promoter P_{lac}. The cultures were incubated for 4 h at 37° C., then centrifuged and prepared for SDS-PAGE analysis. To proof the expression of MutL, Western Blot analysis was done using AP conjugated anti-His-tac antibodies (data not shown).

3. Strain Construction

P1ᵥᵢᵣ lysates were prepared from E. coli strain dapA::kn to infect bacterial cells from the E. coli strains MG1655, MG1655dinB10, MG1655dnaE911zae::cm, MG1655dinB10 dnaE911 zae::cm, MG1655mutL::tet, MG1655dinB10 mutL::tet, MG1655dnaE911zae::cm mutL::tet, MG1655dinB10 dnaE911zae::cm mutL::tet. Obtained single colonies were purified, tested and isolated strains stocked.

4. Growth Studies

a). Reversion of Point Mutations:

Bacterial strain AT997 (dapA15) and MG1655dinB10 dapA::kn were transformed with plasmids pTrcHis2/lacZ and pSU19dapA15, or plasmids pmutL and pSU19dapA15. Single colonies obtained were purified and inoculated in LB containing 100 μg/ml ampicillin; 30 μg/ml chlormaphenicol and 50 μg/ml DAP overnight at 37° C.

b) Reversion of Frameshift Mutations:

Bacterial strain AT997 (dapA15; DapA⁻) was transformed with plasmids pSU40 and pdapAIF, plasmids pSU40mutL and pdapAIF, plasmids pSU40 and pdapA+1, plasmids pSU40mutL and pdapA+1, plasmids pSU40 and pdapA+2, or plasmids pSU40mutL and pdapA+2. Obtained single colonies were purified and inoculated in LB containing 100 μg/ml ampicillin; 50 μg/ml kanamycin and 50 μg/ml DAP overnight at 37° C.

20 μl of overnight cultures of bacteria cells were inoculated in 10 ml LB containing 100 μg/ml ampicillin, 50 μg/ml chloramphenicol and 50 μg/ml DAP at 30° C. During the exponential growth phase, IPTG was added at a final concentration of 1 mM to enhance the expression of MutL from P_{lac} promoter. The cultures were incubated one hour at 30° C. then the temperature was shifted to 37° C. and the cultures were kept overnight at 37° C.

Finally, cultures were centrifuged, 9 ml of media were removed, remaining cells diluted and plated out on agar plates containing 100 μg/ml ampicillin plus 30 μg/ml chloramphenicol or 100 μg/ml ampicillin plus 30 μg/ml chloramphenicol plus 50 μg/ml DAP and incubated for five days at 30° C.

The values for reversion of dapA mutations in wild-type and MutL-overexpressing strains are shown in table 5 and FIG. 6 showing the suppression of mutability via the expression of MutL.

TABLE 5
dap⁻/dap⁺ values in wild-type and MutL overexpressing
strains

| | | dap⁻/ |
|---|---|---|
| Strain | Plasmid | dap⁺ |
| 1-Top10 (wt) | | 0.8 |
| | | 2 |
| 2-AT997 (dapA15) | | 0.0 |
| | | 0 |
| 3-MG1655dinB10 | | 0.0 |
| dapA::kn | | 0 |
| 4-AT997 (dapA15) | pdapAIF, pSU40 | 0.8 |
| | | 1 |
| 5-AT997 (dapA15) | pdapAIF, pSU40mutL | 0.7 |
| | | 2 |
| 6-AT997 (dapA15) | pdapA + 1, pSU40 | 0.0 |
| | | 9 |
| 7-AT997 (dapA15) | pdapA + 1, pSU40mutL | 0.0 |
| | | 000 |
| | | 98 |
| 8-AT997 (dapA15) | pdapA + 2, pSU40 | 0.0 |
| | | 0 |
| 9-AT997 (dapA15) | pdapA + 2, pSU40mutL | 0.0 |
| | | 0 |
| 10-MG1655dinB10 | pSU19dapA (AT997); | 0.5 |
| dapA::kn | pTrcHis2/lacZ | 7 |
| 11-MG1655dinB10 | pSU19dapA (AT997); pmutL | 0.0 |
| dapA::kn | | 51 |

Control strains (1-3) Top10 (wt; DapA⁺), AT997 (dapA15; DapA⁻), and MG1655depA::kn (DapA⁻) show the expected dap⁻/dap⁺ values: approximately 1 for the wild-type strain (growth in the absence of DL-diaminopimelic) and 0 for the dapA⁻ strains (no growth in the absence of DL-diaminopimelic).

The two strains AT997 (dapA15) pdapAIF pTrc (4) and AT997 (dapA15) pdapAIF pmutL (5) show the expected wild-type phenotype as the functional dapA gene is located on the plasmid pdapAIF. Frameshift mutants (6,8) or point mutations of plasmid encoded dapA in a dapA⁻ (10) host should suppress the cell growth in the absence of DAP, while reversion of the introduced mutations confer growth in the absence of DAP (7,11).

Summary and conclusion: In both cases (frameshift and point mutation) in the absence of MutL a higher reversion of mutation was observed. For the frameshift mutation the dap⁻/dap⁺ value drops up to a factor of 1000 and for the point mutation up to 10, meaning that the expression of MutL suppresses the reversion of mutations and thereby acting against the fixation of spontaneous mutations in the chromosome.

### EXAMPLE III

Expression of MutL during “fermentation conditions”

Mutability of producer strain JM83 pXX7, carrying a G-CSF expression vector controlled by tryptophan, was analysed by A) Calculation of the spontaneous rate of mutation by plating out the cells on rifampicilin, B) Calculation of the rate of spontaneous mutation by plating out the cells on phosphomycin and C) Introduction of reporter genes into producer plasmid pXX7 (encodes G-CSF).

To B) Rifampicilin was replaced by phosphomycin, as resistance to phosphomycin develops more rapidly in E. coli under experimental conditions. Phosphomycin is a cell wall inhibitor used mainly for the treatment of uncomplicated lower urinary tract infections.

Experimental Outline

1 Cloning

a) Cloning of a Chloramphenicol Cassette into pXX7:

DNA prepared from plasmid pSU19 was used to amplify the DNA fragment encoding the chloramphenicol resistance by primers 11 (SEQ ID No. 11) and 12 (SEQ ID No. 12) (table 6). These PCR fragments were digested and cloned into the plasmids pXX7 as a BbvcI-AhdI fragment to yield plasmids pXX7cm. The strategy for generating plasmid pXX7cm is shown in FIG. 7A. To simplify the selection process only the −10 region of the “Shine Dalgarno” sequence was cloned.

PCR conditions are as follows (temperature in ° C./time in min): (98/10) (96/0/75; 55/0.50; 72/1)₃₅ (72/10); Herculase.

TABLE 6

Primers used for amplification a DNA
fragment encoding the chloramphenicol
resistance gene

| | |
|---|---|
| primer | Sequence |

| | | |
|---|---|---|
| 11 | cmBbvCl | AACCCTCAGCATAATGAAATAAGATCACTACCGG |
| | hin | |
| 12 | CmAhdl- | CAAGACGATCTCGTCAAGATCATCTTATTAATCAGA |
| | her | TAA |

b) Cloning of a Silent tetA Gene into Plasmid pXX7:

The selection cartridge of plasmid pGBG1 was isolated and cloned into plasmid pXX7 as an MslI-SmaI fragment to yield plasmid pXX7tet (blank-blank ligation). The strategy for generating plasmid pXX7tet is shown in FIG. 7B.

Plasmid pGBG1 was dedicated to the isolation and characterization of mobile elements and other spontaneous mutations in a wide variety of gram-negative bacteria (Schneider et al., Plasmid 44, 201-207 (2000)). The selection cartridge, containing the mutagenesis target, is composed of a silent tetA gene under control of the P_{R} promotor of bacteriophage λ, which is repressed by the λ Cl repressor. Spontaneous mutations (point mutations, deletions, and insertions) that either inactivate cl or eliminate the binding site of CI will derepress P_{R} and therefore trigger expression of tetA. The mutagenesis target is ca. 1 kb long. In this case, expression of MutL should decrease the rate of mutations and therefore reduce the tetracycline resistance in a given host strain.

2. Growth Studies

A) Determination of the Spontaneous Mutation Frequency (SMF) in E. coli by Plating Out the Cells on Rifampicilin

Bacterial strain JM83 pXX7 was transformed with plasmid pTrcHis2/lacZ (control plasmid) and pmutL. Single colonies obtained were purified and the cells inoculated in LB containing 50 μg/ml kanamycin and if required 100 μg/ml ampicillin overnight at 30° C. 3 ml of overnight cultures of these bacteria were inoculated in 100 ml RMG containing 50 μg/ml kanamycin and if required 100 μg/ml ampicillin at 30° C. During the exponential growth phase, IPTG was added at a final concentration of 1 mM to enhance the expression of MutL from P_{lac} promoter of plasmid pmutL, and tryptophane was added at a final concentration of 100 μg/ml to induce the expression of G-CSF from the plasmid pXX7. One hour after induction the inoculation temperature was shifted from 30° C. to 37° C. and then the cells were kept overnight at 37° C.

The cells were centrifuged and the media volume reduced to 10 ml.

Finally, cultures were diluted and plated out on A) LBA plates, B) RMG plates and C) M9 plates containing:
a) 100 μg/ml ampicillin and if required 50 μg/ml kanamycin
b) 100 μg/ml ampicillin, if required 50 μg/ml kanamycin and 100 μg/ml rifampicin
   and incubated for three days at 30° C.

The values for spontaneous mutation frequencies to rifampicin resistance of strains JM83 pXX7, JM83 pXX7 pTrc and JM83 pXX7 pmutL are shown in FIG. 8. Producer strain JM83 pXX7 shows a SMF value of about 0.45. This SMF value remains by additional introduction of the “empty” vector pTrc into the producer strain. But further introduction of pmutL into JM83 pXX7 causes a reduction of the SMF value when MutL is overexpressed by a factor of about 2.

B) Determination of the Spontaneous Mutation Frequency (SMF) in E. coli by Plating Out the Cells on Phosphomycin

Bacterial strain JM83 pXX7 was transformed with plasmid pTrcHis2A and pmutL. Single colonies obtained were purified and for each strain (JM83 pXX7, JM83 pXX7 pTrcA and JM83 pXX7 pmutL) 20 independent, unique cells inoculated in LB containing 50 μg/ml kanamycin and if required 100 μg/ml ampicillin overnight at 30° C.

10 μl overnight cultures of these bacteria were inoculated in 10 ml RMG containing 50 μg/ml kanamycin and if required 100 μg/ml ampicillin at 30° C. During the exponential growth phase, IPTG was added at a final concentration of 1 mM to enhance the expression of MutL from P_{lac} of plasmid pmutL, and tryptophane was added at a final concentration of 100 μg/ml to induce the expression of G-CSF from Pₜᵣₚ of plasmid pXX7. One hour after induction the inoculation temperature was shifted from 30° C. to 37° C. and then the cells were kept overnight at 37° C.

The cells were centrifuged and the media volume reduced to 1 ml.

Finally, cultures were diluted and plated out on LB agar plates containing:
a) 100 μg/ml ampicillin and if required 50 μg/ml kanamycin
b) 100 μg/ml ampicillin, if required 50 μg/ml kanamycin and 30 μg/ml phosphomycin.
   and incubated for three days at 30° C.

The values for spontaneous mutation frequencies to phosphomycine resistance of strains JM83 pXX7, JM83 pXX7 pTrc and JM83 pXX7 pmutL are shown in FIG. 9. Producer strain JM83 pXX7 shows a SMF value of about 4.9. This SMF value remains by an additional introduction of the “empty” vector pTrc into the producer strain. But further introduction of pmutL into JM83 pXX7 causes a reduction of the SMF value when MutL is overexpressed of about a factor 3.

Summary and conclusion: Overexpression of MutL decreases mutability in the G-CSF producer strain around a factor of 3 and heightens cellular viability up to a factor of 10³.

C) Determination of Spontaneous Mutation in E. coli Strain JM83pXX7 by the Introduction of Reporter Genes

a) Expression of G-CSF

b) Expression and Suppression of G-CSF

As G-CSF activity could not be monitored, reporter genes were introduced into plasmid pXX7.

Mutagenesis was assayed in two different systems. In the first system the chloramphenicol resistance gene, which is controlled by a weak promoter, is used. Spontaneous mutations such as point mutations, deletions, and insertions, that deactivate the expression of the cm gene will reduce the chloramphenicol resistance. Absence of MutL should be reported as a reduction of the ratio of spontaneous mutations that confers resistance to chloramphenicol to the total number of viable cells in the culture. In the second system a cassette is used, that contains a silent tetA gene under the control of the P_{R} promotor of bacteriophage λ, and the λcl repressor, which prevents expression of tetA. Spontaneous mutations such as point mutations, deletions, and insertions, that inactivate cl or eliminate the cl binding site will lead to a derepresson of P_{R} and therefore trigger expression of tetA. Expression of MutL should be reported as a reduction of the ratio of spontaneous mutations that confers resistance to tetracycline to the total number of viable cells in the culture.

a) Expression of G-CSF

Bacterial strain JM83 was transformed with plasmids pXX7cm, pXX7cm pTrcHis2/lacZ, pXX7cm pmutL, pXX7tet, and pXX7tet pTrcHis2/lacZ and pXX7tet pmutL. Single colonies obtained were purified and cells inoculated in LB containing 50 μg/ml kanamycin and if required 100 μg/ml ampicillin overnight at 30° C.

10 μl overnight cultures of these bacteria were inoculated in 10 ml RMG containing, 50 μg/ml kanamycin and if required 100 μg/ml ampicillin at 30° C. During the exponentially growing phase, IPTG was added at a final concentration of 1 mM to enhance the expression of MutL from P_{lac} of plasmid pmutL, and tryptophane was added at a final concentration of 100 μg/ml to induce the expression of G-CSF from Pₜᵣₚ of plasmid pXX7. One hour after induction the inoculation temperature was shifted from 30° C. to 37° C. and then the cells were kept overnight at 37° C.

The cells were centrifuged and the media volume reduced to 1 ml.

Finally, cultures were diluted and plated out on LB agar plates containing different antibiotics (shown in table 7) and incubated for four days at 30° C.

TABLE 7
Used antibiotics

| Strain | Antibiotics | | |
|---|---|---|---|
| JM83 pXX7 | 50 μg/ml | 50 μg/ml | 50 μg/ml |
| | kanamy-cin | kanamycin | kanamycin |
| | | 30 μg/ml | 12.5 μg/ml |
| | | chloram- | tetracy-cline |
| | | phenicol | |
| JM83 pXX7 pTrcA | 50 μg/ml | 50 μg/ml | 50 μg/ml |
| | kanamycin | kanamycin | kanamycin |
| | 100 μg/ml | 30 μg/ml | 12.5 μg/ml |
| | ampicillin | chloramphenicol | tetracycline |
| | | 100 μg/ml | 100 μg/ml |
| | | ampicillin | ampicillin |
| JM83 pXX7 pmutL | 50 μg/ml | 50 μg/ml | 50 μg/ml |
| | kanamycin | kanamycin | kanamycin |
| | 100 μg/ml | 30 μg/ml | 12.5 μg/ml |
| | ampicillin | chloramphenicol | tetracycli-ne |
| | | 100 μg/ml | 100 μg/ml |
| | | ampicillin | ampicillin |
| JM83 pXX7cm | 50 μg/ml | 50 μg/ml | |
| | kanamy-cin | kanamycin | |
| | | 30 μg/ml | |
| | | chloramphe-nicol | |
| JM83 pXX7cm | 50 μg/ml | 50 μg/ml | |
| pTrcA | kanamycin | kanamycin | |
| | 100 μg/ml | 30 μg/ml | |
| | ampicillin | chloramphenicol | |
| | | 100 μg/ml | |
| | | ampicillin | |
| JM83 pXX7cm | 50 μg/ml | 50 μg/ml | |
| pmutL | kanamycin | kanamycin | |
| | 100 μg/ml | 30 μg/ml | |
| | ampicillin | chloramphenicol | |
| | | 100 μg/ml | |
| | | ampicillin | |
| JM83 pXX7tet | 50 μg/ml | | 50 μg/ml |
| | kanamycin | | kanamycin |
| | | | 12.5 μg/ml |
| | | | tetracycli-ne |
| JM83 pXX7tet | 50 μg/ml | | 50 μg/ml |
| pTrcA | kanamy-cin | | kanamycin |
| | 100 μg/ml | | 12.5 μg/ml |
| | ampicil-lin | | tetracycli-ne |
| | | | 100 μg/ml |
| | | | ampicillin |
| JM83 pXX7tet | 50 μg/ml | | 50 μg/ml |
| pmutL | kanamy-cin | | kanamycin |
| | 100 μg/ml | | 12.5 μg/ml |
| | ampicil-lin | | tetracycli-ne |
| | | | 100 μg/ml |
| | | | ampicillin |

The values for spontaneous mutation frequencies in the chloramphenicol resistance reporter gene for strains JM83 pXX7cm, JM83 pXX7cm pTrc and JM83 pXX7cm pmutL are shown in FIG. 10. For the two strains JM83 pXX7cm and JM3 pXX7cm pTrc the mutability values were 0.28 or 0.35 (Δ1.25). Expression of MutL decreases the value of mutability to 0.49 (Δ1.75). This means that the expression of MutL suppresses the conversion of chloramphenicol resistance to chloramphenicol sensitivity and thereby acting against the fixation of spontaneous mutation in the chromosome.

The values for spontaneous mutation frequencies in the silent tetA reporter gene for strains JM83 pXX7tet, JM83 pXX7tet pTrc and JM83 pXX7tet pmutL are shown in FIG. 11. For the two strains JM83 pXX7tet and JM83 pXX7tet pTrc the mutability values were 0.43 or 0.39 (Δ1.1). Expression of MutL drops the value of mutability to 0.25 (Δ1.72). This means that the expression of MutL suppresses the conversion of the tetracycline sensitivity to tetracycline resistance and thereby acting against the fixation of spontaneous mutations in the chromosome.

b) Expression and Suppression of G-CSF

Wild-type bacteria strain JM83 was transformed with plasmids pXX7cm, pXX7cm pTrcHis2/lacZ, pXX7cm pmutL, pXX7tet, and pXX7tet pTrcHis2/lacZ and pXX7tet pmutL. Single colonies obtained were purified and cells inoculated in LB containing 50 μg/ml kanamycin and if required 100 μg/ml ampicillin overnight at 30° C.

10 μl overnight cultures of these bacteria were inoculated in 10 ml RMG containing 50 μg/ml kanamycin and if required 100 μg/ml ampicillin at 30° C. During the exponential growth phase, IPTG was added at a final concentration of 1 mM to enhance the expression of MutL from the P_{trc} promoter of plasmid pmutL. The cultures were bisected and tryptophane was once added at a final concentration of 100 μg/ml to induce the expression of G-CSF from the Pₜᵣₚ promoter of plasmid pXX7. One hour after induction the inoculation temperature was shifted from 30° C. to 37° C. and then the cells were kept overnight at 37° C.

The cells were centrifuged and the media volume reduced to 1 ml.

Finally, cultures were diluted and plated out on LB agar plates containing different antibiotics (see table 7) and incubated overnight at 37° C.

The mutability of the modified producer strain JM83 pXXcm during fermentation conditions is represented in FIG. 12. In the absence of G-CSF, the measured frequency of chloramphenicol resistance shifts between 50 and 55 for JMV83 pXX7cm, JM83 pXX7cm pTrc and JM83 pXX7cm pmutL. Induction of G-CSF drops the values for JM83 pXX7tet and JM83 pXX7tet pTrc up to a factor of 3. But for strain JM83 pXX7 pmutL the chloramphenicol resistance remains when G-CSF is expressed.

The expression of MutL suppresses the conversion of chloramphenicol resistance to chloramphenicol sensitivity. MutL acts against the fixation of spontaneous mutations in the chromosome.

FIG. 13 shows the significant increase of mutability in the modified producer strain JM83 pXX7 during fermentation conditions. But for strain JM83 pXX7 pmutL that expresses plasmid-encoded MutL the tetracycline resistance stays constant when G-CSF is expressed. Expression of MutL suppresses the conversion of tetracycline resistance to tetracycline sensitivity and acting against the fixation of spontaneous mutations in the chromosome.

Conclusion and summary: The results obtained show that an over-expression of MutL decreases mutability in the G-CSF fermentation strain during the production of G-CSF around 3 fold. Furthermore it was found that the overexpression of MutL heightens cellular viability by a factor of 10³.

The induction of G-CSF heightens the rate of spontaneous mutations in the tested producer strains about a factor 2 to 3.

1. Process for reducing the spontaneous mutation frequencies in a cell or an organism by introducing at least two mutations, whose combined actions lead to at least two enhanced cellular DNA repair mechanisms, into the cell or organism.

2. Process for producing a cell or an organism with reduced spontaneous mutation frequencies by introducing at least two mutations, whose combined actions lead to at least two enhanced cellular DNA repair mechanisms, into at least one cell of the organism and regenerating the organism therefrom if the organism is a multicellular organism.

3. Process according to claim 1, wherein the cell is a prokaryotic cell.

4. Process according to claim 3, wherein the prokaryotic cell is a cell of an archaebacterium or a eubacterium.

5. Process according to claim 4, wherein the cell of the eubacterium is a cell of a gram-positive or a gram-negative bacterium.

6. Process according to claim 1, wherein the cell is a eukaryotic cell.

7. Process according to claim 6, wherein the eukaryotic cell is a fungal cell, animal cell or plant cell.

8. Process according to claim 1, wherein the organism is a fungus, animal or plant.

9. Process according to claim 1, wherein the combined action of the at least two mutations leads to an enhanced capability of at least two cellular DNA repair mechanisms to repair spontaneously occurring mutations.

10. Process according to claim 9, wherein the capability of the mismatch repair system, the post-replicative repair system and/or the SOS repair system to repair spontaneously occurring mutations is enhanced.

11. Process according to claim 1, wherein the at least two mutations are selected from a mutation leading to an upregulation of the expression of the MutL protein or a homologous protein thereof, a mutation leading to an upregulation of the expression of the MutS protein or a homologous protein thereof, an antimutator allele of a gene encoding DNA polymerase IV or a homologous protein thereof and an antimutator allele of a gene encoding a subunit of DNA polymerase III or a homologous protein thereof.

12. Process according to claim 11, wherein the upregulation of the expression of MutL, MutS or a homologous protein thereof is achieved by introducing a vector within the cell, wherein the vector comprises the mutL gene, a gene encoding a homologous protein of MutL, the mutS gene or a gene encoding a homologous protein of MutS under the functional control of one or more regulatory units allowing an overexpression of MutL, MutS or a homologous protein thereof.

13. Process according to claim 12, wherein the vector is a multi-copy plasmid.

14. Process according to claim 11, wherein the regulatory unit is an inducible or constitutive promoter.

15. Process according to claim 11, wherein the upregulation of the expression of MutL, MutS or a homologous protein thereof is achieved by introducing one or more additional copies of the respective mut gene under the functional control of one or more regulatory units into the chromosome(s) of the host cell and/or by introducing of one or more mutations into the regulatory units controlling the expression of the native Mut Protein such that the production of the respective Mut protein in increased in comparison to a corresponding wild-type cell.

16. Process according to claim 11, wherein the antimutator allele of the gene encoding DNA polymerase IV is dinB10.

17. Process according to claim 11, wherein the antimutator allele of the gene encoding the subunit of DNA polymerase III is dnaE911.

18. Process according to claim 1, wherein the combined action of dinB10 and dnaE911 reduces the spontaneous mutation frequencies in comparison to a wild-type cell or wild-type organism at least 10-fold.

19. Process according to claim 1, wherein the combined action of dinB10, dnaE911 and overexpressed mutL reduces the spontaneous mutation frequencies in comparison to a wild-type cell or wild-type organism at least 50-fold.

20. Process according to claim 1, wherein the combined action of the at least two mutations leads to an enhanced cellular viability.

21. Cell with reduced spontaneous mutation frequencies and/or enhanced cellular viability and obtainable by a process according to claim 1, wherein the cell comprises at least two mutations, whose combined actions lead to at least two enhanced cellular DNA repair mechanisms.

22. Cell according to claim 21, wherein the cell is a bacterial, fungal, plant or animal cell.

23. Organism with reduced spontaneous mutation frequencies and obtainable by a process according to claim 1, wherein the cells of the organism comprise at least two mutations, whose combined actions lead to at least two enhanced cellular DNA repair mechanisms.

24. E. coli MG1655dinB10 containing plasmid pmutL (DSM 17016).

25. E. coli MG1655dinB10 mutL::tet containing plasmid pmutL (DSM 17017).

26. E. coli MG1655 dnaE zae::cm containing plasmid pmutL (DSM 17018).

27. E. coli MG1655 dnaE zae::cm mutL::tet containing plasmid pmutL (DSM 17019).

28. E. coli MG1655dinB10 dnaE zae::cm (DSM 17015).

29. E. coli MG1655dinB10 dnaE zae::cm mutL::tet (DSM 17014).

30. E. coli MG1655dinB10 dnaE zae::cm containing plasmid pmutL (DSM 17020).

31. E. coli MG1655dinB10 dnaE zae::cm mutL::tet containing plasmid pmutL (DSM 17021).

32. Process for the generation of an expression system for a protein wherein the amino acid sequence of the protein is stabilized against spontaneously occurring mutations comprising:
a) inserting a nucleic acid sequence encoding the protein into the genome of a host cell, that contains at least two mutations whose combined actions lead to an enhanced capability of at least two cellular DNA repair mechanisms to repair spontaneously occurring mutations, under the functional control of one or more regulatory units allowing an inducible or constitutive expression of the protein, or
b) inserting a nucleic acid sequence encoding the protein into a vector under the functional control of one or more regulatory units allowing an inducible or constitutive expression of the protein and transferring the vector into a host cell, that contains at least two mutations whose combined actions lead to an enhanced capability of at least two cellular DNA repair mechanisms to repair spontaneously occurring mutations and
c) culturing and/or maintaining the host cell in an appropriate medium.

33. Process for the production of a protein wherein the amino acid sequence of the protein is stabilized against spontaneously occurring mutations comprising:
a) inserting a nucleic acid sequence encoding the protein into the genome of a host cell, that contains at least two mutations whose combined actions lead to an enhanced capability of at least two cellular DNA repair mechanisms to repair spontaneously occurring mutations, under the functional control of one or more regulatory units allowing an inducible or constitutive expression of the protein, or
b) inserting a nucleic acid sequence encoding the protein into a vector under the functional control of one or more regulatory units allowing an inducible or constitutive expression of the protein and transferring the vector into a host cell, that contains at least two mutations whose combined actions lead to an enhanced capability of at least two cellular DNA repair mechanisms to repair spontaneously occurring mutations,
c) culturing the host cell in an appropriate medium under conditions allowing the expression of the protein, and
d) isolating the protein expressed.

34. Process according to claim 33, wherein the protein is isolated from the medium.

35. Process according to claim 33, wherein the protein is extracted from the host cell.

36. Process according to claim 32, wherein the protein is a therapeutically usable protein, in particular a cytokine or a growth factor.

37. Process according to claim 32, wherein the vector is a plasmid, bacteriophage or cosmid.

38. Process according to claim 32, wherein the regulatory unit is a promoter, a ribosome binding site, an enhancer, a silencer and/or a 3′-transcription terminator.

39. Process according to claim 32, wherein the nucleic acid sequence encoding the protein is functionally linked to a leader sequence directing the transport of the protein expressed to a cell organelle, a cell compartment, the extracellular space or into the medium.

40. Process for the production of a fermentation product by cultivating a cell producing the fermentation product and/or at least one enzyme involved in the formation of the fermentation product in a medium wherein the genome of the cell is stabilized against spontaneously occurring sequence changes by at least two mutations whose combined actions lead to an enhanced capability of at least two cellular DNA repair mechanisms to repair spontaneously occurring mutations.

41. Process according to claim 40, wherein the fermentation product is a nucleic acid, a nucleoside, a nucleotide, an amino acid, a protein, an acid, a carbohydrate, a vitamin, an antibiotic or an alkaloid.

42. Process according to claim 32, wherein the capability of the mismatch repair system, the proof-reading function and/or the SOS repair system to repair spontaneously occurring mutations is enhanced.

43. Process according to claim 32, wherein the at least two mutations are selected from a mutation leading to an upregulation of the expression of the MutL protein or a homologous protein thereof, a mutation leading to an upregulation of the expression of the MutS protein or a homologous protein thereof, an antimutator allele of a gene encoding DNA polymerase IV or a homologous protein thereof and an antimutator allele of a gene encoding a sub-unit of DNA polymerase III or a homologous protein thereof.

44. Process according to claim 43, wherein the upregulation of the expression of MutL, MutS or a homologous protein thereof is due to the presence of a vector within the cell, wherein the vector comprises the mutL gene, a gene encoding a homologous protein of MutL, the mutS gene or a gene encoding a homologous protein of MutS under the functional control of one or more regulatory units allowing an overexpression of MutL, MutS or the homologous protein thereof.

45. Process according to claim 43, wherein the upregulation of the expression of MutL, MutS or a homologous protein thereof is achieved by introducing one or more additional copies of the respective mut gene under the functional control of one or more regulatory units into the chromosome(s) of the host cell and/or by introducing of one or more mutations into that regulatory units controlling the expression of the native Mut Protein such that the production of the respective Mut protein is increased in comparison to a corresponding wild-type cell.

46. Process according to claim 43, wherein the antimutator allele of the gene encoding DNA polymerase IV is dinB10.

47. Process according to claim 43, wherein the antimutator allele of the gene encoding the subunit of DNA polymerase III is dnaE911.

48. Process according to claim 32, wherein the combined action of dinB10 and dnaE911 reduces the spontaneous mutation frequencies in comparison to a wild-type cell at least 10-fold.

49. Process according to claim 32, wherein the combined action of dinB10, dnaE911 and overexpressed mutL reduces the spontaneous mutation frequencies in comparison to a wild-type cell at least 50-fold.

50. Process according to claim 32, wherein the combined action of the at least two mutations leads to an enhanced cellular viability.

51. Process according to claim 32, wherein the cell is a prokaryotic or eukaryotic cell.

52. Process according to claim 51, wherein the cell is a cell of a gram-positive or gram-negative bacterium.

53. Process according to claim 51, wherein the cell is a fungal cell, animal cell or plant cell.

54. Process according to claim 32, wherein the cell is a cell obtainable by a process according to claim 1.

55. Process according to claim 32, wherein the cell is cultivated in a liquid medium.

56. Process according to claim 55, wherein the cell is cultivated in a continuous culture or in a batch culture.

57. Process according to claim 32, wherein the cell is immobilized.

58. Process according to claim 32, wherein the cell is cultivated on a solid or semi-solid medium.

59. Process according to claim 40, wherein the fermentation product is isolated from the cell.

60. Process according to claim 40, wherein the fermentation product is isolated from the medium.

61. Protein obtainable by a process according to claim 32.

62. Fermentation product is obtainable by a process according to claim 40.


SEQUENCE LISTING (12 sequences)

SEQ ID NO: 1 - DNA, 37 bp, Artificial Sequence
  Description of Artificial Sequence primer
catgccatggcgccaattcaggtcttaccgccacaac

SEQ ID NO: 2 - DNA, 37 bp, Artificial Sequence
  Description of Artificial Sequence primer
ccgctcgagcctcatctttcagggcttttatcgccgg

SEQ ID NO: 3 - DNA, 38 bp, Artificial Sequence
  Description of Artificial Sequence primer
cgcggatccgagtgcaatagaaaatttcgacgcccata

SEQ ID NO: 4 - DNA, 38 bp, Artificial Sequence
  Description of Artificial Sequence primer
ccgctcgagccaccaggctcttcaagcgataaatccac

SEQ ID NO: 5 - DNA, 35 bp, Artificial Sequence
  Description of Artificial Sequence primer
cgcggatccgttcacgggaagtattgtcgcgattg

SEQ ID NO: 6 - DNA, 36 bp, Artificial Sequence
  Description of Artificial Sequence primer
cgcggatccgcttcacgggaagtattgtcgcgattg

SEQ ID NO: 7 - DNA, 37 bp, Artificial Sequence
  Description of Artificial Sequence primer
cgcggatccgcgttcacgggaagtattgtcgcgattg

SEQ ID NO: 8 - DNA, 35 bp, Artificial Sequence
  Description of Artificial Sequence primer
ccgctcgagccagcaaaccggcatgcttaagcgcc

SEQ ID NO: 9 - DNA, 39 bp, Artificial Sequence
  Description of Artificial Sequence primer
ccggaattccgggcatacaacaatcagaacggttctgtc

SEQ ID NO: 10 - DNA, 39 bp, Artificial Sequence
  Description of Artificial Sequence primer
cccaagcttgggcgaaacacccgcaacctttgccaggcg

SEQ ID NO: 11 - DNA, 34 bp, Artificial Sequence
  Description of Artificial Sequence primer
aaccctcagcataatgaaataagatcactaccgg

SEQ ID NO: 12 - DNA, 39 bp, Artificial Sequence
  Description of Artificial Sequence primer
caagacgatctcgtcaagatcatcttattaatcagataa